(19) United States
(12) Patent Application Publication
(10) Pub. No.: US 2026/0193319 A1
(43) Pub. Date: Jul. 9, 2026

(54) PLATELET-BINDING PROTEINS AND CONJUGATES THEREOF, PARTICLES COMPRISING THEM AND USES THEREOF

(71) Applicant: Haima Therapeutics, LLC, Cleveland, OH (US)

(72) Inventors: Christa Pawlowski, Solon, OH (US); Michael Bruckman, Rocky River, OH (US); Andrew Ditto, Hudson, OH (US); Noah Duffy, South Pasadena, CA (US)

(21) Appl. No.: 19/130,803

(22) PCT Filed: Nov. 17, 2023
(86) PCT No.: PCT/US23/80225

§ 371 Date: May 16, 2025

Related U.S. Application Data
(60) Provisional application No. 63/426,521, filed on Nov. 18, 2022.

(51) Int. Cl. C07K 14/75; A61K 47/54; A61K 47/60; A61K 47/64; A61K 47/69; A61P 7/04

(52) U.S. Cl. CPC C07K 14/75; A61K 47/548; A61K 47/60; A61K 47/64; A61K 47/6927; A61P 7/04

(57) ABSTRACT

The present application relates to platelet binding peptides and their conjugates, as well as particles comprising these and other peptides and conjugates. In addition, compositions and methods for using these peptides and particles are provided.

### RELATED APPLICATIONS

This application claims the benefit of priority to U.S. Provisional Patent Application Ser. No. 63/426,521, filed on Nov. 18, 2022, which is hereby incorporated by reference in its entirety.

### GOVERNMENT SUPPORT

This invention was made with government support under Grant Numbers 1951301, awarded by the National Science Foundation. The government has certain rights in the invention.

### BACKGROUND

Managing uncontrolled bleeding requires a multifactorial approach. Uncontrolled bleeding occurs in a variety of clinical indications including (but not limited to) traumatic injury, surgery, thrombocytopenia, and postpartum hemorrahge. Currently, the gold standard of care in these indications is transfusion of human donor platelets in conjunction with other blood product therapies, such as packed red blood cells (RBCs), plasma, and/or whole blood. Platelets are the blood cells primarily responsible for forming the primary hemostatic clot at the bleeding injury site and also for facilitating secondary hemostasis to secure and stabilize the clot for hemorrhage control. Overwhelming evidence from previous clinical studies shows the benefit of donor platelet transfusion to prevent or treat bleeding. However, donor platelets have limited availability and portability, special storage requirements, high contamination risks, and very short shelf life, which present logistical challenges for their widespread accessibility and sustained utilization. Furthermore, blood transfusions are not without considerable risks, including nosocomial infection, immunosuppression, transfusion-related acute lung injury, and even death.

Non-platelet alternatives, including red blood cells modified with the platelet-binding Arg-Gly-Asp (RGD) sequence, fibrinogen-coated microcapsules based on albumin, and liposomal systems, have been studied as coagulants, but these products suffer from toxicity, thrombosis, and limited efficacy. RGD peptides can bind to beta 3 integrins, including glycoprotein (GP) IIb-IIIa on platelets. Furthermore, certain cyclic RGD peptides have selective and potent binding to the activated GPIIb/IIIa protein over the inactive form or other beta 3 integrins. The original cyclic peptide referenced here (first reported by Cheng et al., J. Med. Chem. 1994, 37, 1, 1-8) and further modified to allow bioconjugation to lipids, is cyclized with a Cys-Cys disulfide chemistry. However, this cyclic RGD peptide is not chemically stable.

Further, non-platelet alternatives with surface decoration containing multiple peptides or proteins can render multiple hemostatic functionalities such as binding to various injury-site relevant cells (e.g. platelets) and proteins (e.g. collagen and von Willebrand Factor).

### SUMMARY

The present invention is based, at least in part, on the discovery that the chemical stability of a cyclic RGD peptide was greatly improved by modifying the peptide chemistry to replace the terminal Cys-Cys cyclization (disulfide bond) with beta-alanine mediated N—C cyclization. The cyclization bond, the sequence around the RGD motif, the number of amino acids, and the size of the ring structure have changed in the new chemistry. Despite these changes, the peptide has an unexpected 2-fold increase in its anti-thrombotic potency (IC₅₀=0.13 μM via platelet aggregation in human PRP/ADP) from the peptide structure reported in Cheng et al. (IC₅₀=0.22 μM) (see U.S. Pat. No. 9,107,845, which is hereby incorporated by reference in its entirety). Furthermore, this peptide structure is also amenable to chemical coupling to maleimide functionalized lipids, proteins, polymers, etc., through thio-ene coupling to the now free thiol group on the N-terminal cysteine. Depending on the physicochemical properties of these bioconjugates, they can self-assemble into particles. Particles containing the new GPIIb-IIIa peptides alone or in combination with other motifs promote platelet aggregation and can be used as hemostatic agents (as opposed to anti-thrombotic applications as stated above).

The present invention is also based, at least in part, on the discovery that certain peptides, when present on the surface of a synthetic platelet, result in more efficient hemostatic activity (i.e., efficient clotting). Additionally, mole percentages of the peptides disclosed here that decorate a particle can be optimized to significantly increase the hemostatic activity of the particle. In certain embodiments, the disclosure relates to a particle comprising a plurality of platelet binding peptide (PBP) conjugates, von Willebrand factor-binding peptide (VBP) conjugates, or collagen-binding peptide (CBP) conjugates, or a combination thereof, wherein the PBP conjugate is a fibrinogen mimetic peptide (FMP) conjugate or a P-selectin binding peptide conjugate, and wherein the plurality of PBP conjugates, VBP conjugates, and/or CBP conjugates are conjugated to an outer surface of the particle, wherein the PBP conjugates, VBP conjugates, and CBP conjugates, taken together, are present at <5 molar percent of the particle.

In certain embodiments, the disclosure relates to a fibrinogen mimetic peptide (FMP) of Formula (I):

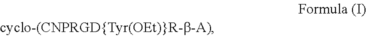

or a salt thereof. In certain embodiments, the disclosure relates to a peptide conjugate comprising a peptide conjugated to a polymer, wherein the peptide is a fibrinogen mimetic peptide is any of the FMPs described herein. In certain embodiments, the disclosure relates to a particle comprising a plurality of platelet binding peptide (PBP) conjugates, wherein the PBP conjugates comprise any of the peptide conjugates described herein. In certain embodiments, the disclosure relates to a pharmaceutical composition comprising any of the fibrinogen mimetic peptides described herein and a carrier. In certain embodiments, the disclosure relates to a pharmaceutical composition comprising any of the peptide conjugates described herein and a carrier. In certain embodiments, the disclosure relates to a method of preventing or inhibiting platelet aggregation in a subject in need thereof, comprising administering to the subject a composition comprising any of the fibrinogen mimetic peptides (FMP) described herein. In certain embodiments, the disclosure relates to a method of preventing or inhibiting platelet aggregation in a subject in need thereof, the method comprising administering to the subject any of the fibrinogen mimetic peptides described herein or any of the pharmaceutical compositions comprising FMPs described herein.

In certain embodiments, the disclosure relates to a pharmaceutical composition comprising any of the particles described herein and a carrier.

In certain embodiments, the disclosure relates to a method of promoting aggregation of activated platelets on a site with exposed vWF and collagen, comprising administering to the site any of the particles described herein, or any of the pharmaceutical compositions comprising particles described herein.

In certain embodiments, the disclosure relates to a method of diminishing bleeding, treating a vascular injury, or promoting hemostasis in a subject, comprising administering to a site of bleeding or a site of vascular injury any of the particles described herein, or any of the pharmaceutical compositions comprising particles described herein.

### BRIEF DESCRIPTION OF THE DRAWINGS

FIGS. 1A-1B show dose dependent inhibition in human platelet final aggregation via light transmission aggregometry in both 1A) cyclo-{Pra}CNPRGD{Tyr(OEt)}RC (FMP1) and 1B) cyclo-(CNPRGD{Tyr(OEt)}R-β-A) (FMP2). FIG. 1C) shows a log-dose response (% aggregation inhibition) curve shows an IC₅₀=536.6 μM for FMP1 and IC₅₀=0.13 μM for FMP2. Statistical significance denoted as *P<0.05, **P<0.01, ***P<0.001, ****P<0.0001.

FIG. 2A shows evidence of 2A) FMP1 dimer/trimerization using MALDI-TOF mass spectrometry, likely due to cyclic disulfide bond breaking and reforming with free sulfhydryls on neighboring peptides, suggesting poor stability. FIG. 2B) shows no evidence of dimer/trimerization of FMP2.

FIGS. 3A-3B show that MALDI-TOF mass spectrometry confirms conjugation of 3A) FMP1 to DSPE-PEG(2000)-Azide and 3B) FMP2 to DSPE-PEG(2000)-maleimide.

FIG. 4 shows synthetic platelet particle effective diameter in nanometers (nm) as measured by dynamic light scattering over 15 weeks storage in buffer at room temperature. Particle 1=synthetic platelet formulation with 15 mole % FMP1, Particle 2=synthetic platelet formulation with 2.5 mole % FMP1, Particle 3=synthetic platelet formulation with 15 mole % FMP2, Particle 4=synthetic platelet formulation with 2.5 mole % FMP2. Particles containing FMP2 are smaller in average than particles with FMP1, possibly due to less particle aggregation.

FIG. 5 shows percent (%) final platelet aggregation (normalized to simulated thrombocytopenic platelet rich plasma [tPRP] group) of synthetic platelet particles containing FMP1 vs. FMP2 peptide. Particles with FMP2 showed enhanced platelet aggregation to a similar degree as particles with FMP1. Particle size (200 nm vs. 100 nm effective diameter measured via DLS) did not affect platelet aggregation potential. NA=no agonist. Error bars for particles containing 15% FMP at 200 nm are n=4.

FIGS. 6A-6E show flow cytometry evaluation of Rhodamine-B-labeled synthetic platelet particle containing 15 mole % FMP2 (Particle 3) vs. FMP1 (Particle 1) binding to activated human washed platelets compared to control particles (CP, liposomes containing no peptide decoration). 6A-6B) Gating for platelet population via forward and size scatter, 6C) Gating for platelets using CD41a positive events, 6D) Histogram of Rhodamine B-positive events to TRAP activated platelets, 6E) Histogram of Rhodamine-B-positive events to ADP activated platelets. 6D-6E) Tables show mean Rhodamine B (YG582-A) and mean platelet activation via CD62P labeling (R660-A) levels.

FIGS. 7A-7Y show representative DLS intensity diameter relative frequency histograms for synthetic platelet particles formulations: 7A) Particle 1, 7B) Particle 2, 7C) Particle 3, 7D) Particle 4, 7E) Particle 5, 7F) Particle 6, 7G) Particle 7, 7H) Particle 8, 7I) Particle 9, 7J) Particle 10, 7K) Particle 11, 7L) Particle 12, 7M) Particle 14, 7N) Particle 15, 7O) Particle 16, 7P) Particle 18, 7Q) Particle 19, 7R) Particle 20, 7S) Particle 21, 7T) Particle 22, 7U) Particle 23, 7V) Particle 24, 7W) Particle 26, 7X) Particle 27, 7Y) Particle 28.

FIGS. 8A-8C show representative Cryo-TEM images and particle size histograms of 8A) Particle 5, 8B) Particle 11, and 8C) Particle 12.

FIG. 9 shows functional analysis of various synthetic platelet formulations (Cy-5 labeled) binding to activated human platelets using flow cytometry. Platelet:Particle ratio ~1:1000.

FIGS. 10A-10B shows platelet aggregation profile of normal (+) and platelet-reduced plasma (−) samples in the presence of various synthetic platelet formulations. Platelet:particle ratio ~1:10. Results reported as 10A) % final aggregation and 10B) % maximum aggregation normalized to the TCP defect without particle condition.

FIGS. 11A-11D show functional analysis of various synthetic platelet formulations using BioFlux shows 11A) platelet binding area (% covered), 11B) platelet binding rate, 11C) particle binding area (% covered), and 11D) particle binding rate to collagen/vWF coated surfaces at low platelet counts under flow. Positive control (+) is platelets at normal counts. Negative control (−) is platelets at low counts with vehicle (buffer).

FIGS. 12A-12B shows 12A) Blood loss volume and 12B) bleeding time after tail transection in thrombocytopenic mice treated with various synthetic platelet formulations (Particles 5, 12, 20, 22, and 28) at various doses 0.1-10 mg/kg. Baseline represents bleeding in mice with normal platelet counts. Negative control (−) is treatment with vehicle (buffer). Positive control (+) is treatment with allogeneic platelets. Statistical significance compared to negative control group denoted as *p<0.05. Number inside bar represents n per group.

### DETAILED DESCRIPTION

It has been determined herein that the chemical stability of the cyclic Arg-Gly-Asp (RGD) peptide was greatly improved by modifying the peptide chemistry to replace the terminal Cys-Cys cyclization (disulfide bond) with beta-alanine mediated N—C cyclization. Accordingly, the present invention relates, in part, to a platelet binding protein (PBP) of Formula (I) and conjugates comprising a PBP of Formula (I). In addition, in certain embodiments, the invention relates to particles comprising
platelet binding proteins (PBPs), for example P-selectin binding peptides (DAEWVDVS (SEQ ID NO: 5)) or fibrinogen mimetic peptides (FMPs), such as FMPs of Formula (I) (also referred to herein as FMP2; cyclo-(CNPRGD{Tyr(OEt)}R-β-C) (SEQ ID NO: 1)), FMP1(cyclo-(CNPRGD{Tyr(OEt)}R-β-C) (SEQ ID NO: 2)), linear RGD (GRGDSP (SEQ ID NO: 3)), and H12 (HHLGGAKQAGDV (SEQ ID NO: 4));
collagen binding peptides (CBPs), for example (GPO)₇ (SEQ ID NO: 8); or
von Willebrand binding proteins (VBPs), for example TRYLRIHPQSWVHQI (SEQ ID NO: 6), or combinations thereof, or their conjugates thereof, or combinations of conjugates thereof. In addition, compositions and methods for using PBPs, CBPs, and VBPs and particles conjugated to these peptides are also provided.

### I. Definitions

The articles “a” and “an” are used herein to refer to one or to more than one (i.e., to at least one) of the grammatical object of the article. By way of example, “an element” means one element or more than one element.

The term “administering” is intended to include routes of administration which allow an agent (such as the compositions described herein) to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions that may detrimentally affect its ability to perform its intended function. The agent may be administered alone, or in conjunction with a pharmaceutically acceptable carrier. The agent also may be administered as a prodrug, which is converted to its active form in vivo. In some embodiments, the agent is orally administered. In other embodiments, the agent is administered through anal and/or colorectal route.

“About” and “approximately” shall generally mean an acceptable degree of error for the quantity measured given the nature or precision of the measurements. Typically, exemplary degrees of error are within 20%, preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

A “conservative substitution” is the substitution of an amino acid with another amino acid with similar physical and chemical properties. In contrast, a “nonconservative substitution” is the substitution of an amino acid with another amino acid with dissimilar physical and chemical properties.

The term “comprise” or variations such as “comprises” or “comprising” will be understood to imply the inclusion of a stated integer (or components) or group of integers (or components), but not the exclusion of any other integer (or components) or group of integers (or components).

As used herein, “homology” is used synonymously with “identity.”

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

A “kit” is any manufacture (e.g., a package or container) comprising at least one reagent, e.g. a probe or small molecule, for specifically detecting and/or affecting the expression of a marker of the present invention. The kit may be promoted, distributed, or sold as a unit for performing the methods of the present invention. The kit may comprise one or more reagents necessary to express a composition useful in the methods of the present invention. In certain embodiments, the kit may further comprise a reference standard. One skilled in the art can envision many such controls, including, but not limited to, common molecules. Reagents in the kit may be provided in individual containers or as mixtures of two or more reagents in a single container. In addition, instructional materials that describe the use of the compositions within the kit can be included.

“Mutants,” “derivatives,” and “variants” of a polypeptide (or of the DNA encoding the same) are polypeptides that may be modified or altered in one or more amino acids (or in one or more nucleotides) such that the peptide (or the nucleic acid) is not identical to the wild-type sequence, but has homology to the wild type polypeptide (or the nucleic acid).

A “mutation” of a polypeptide (or of the DNA encoding the same) is a modification or alteration of one or more amino acids (or in one or more nucleotides) such that the peptide (or nucleic acid) is not identical to the sequences recited herein, but has homology to the wild type polypeptide (or the nucleic acid).

“Particle” as used herein is meant to include particles, spheres, capsules, and other structures having a length or diameter of about 10 nm to about 10 m. For the purposes of this application, the terms “nanosphere,” “nanoparticle,” “nanocapsule,” “microsphere,” “microparticle,” “microcapsule,” and “particle” are used interchangeably.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

A “portion” of a polypeptide means at least about three sequential amino acid residues of the polypeptide. It is understood that a portion of a polypeptide may include every amino acid residue of the polypeptide.

As used herein, a therapeutic that “prevents” a condition refers to a composition that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

A “recombinant polypeptide” is a polypeptide that is produced upon expression of a recombinant polynucleotide.

The term “site” refers to a breach of a surface, for example, the site of an injury, wherein the breach results in von Willebrand Factor and collagen being present at the site.

The term “synergistic effect” refers to the combined effect of two or more agents described herein can be greater than the sum of the separate effects of any one of agents alone.

The terms “subject” refer to either a human or a non-human animal. This term includes mammals such as humans, primates, livestock animals (e.g., bovines, porcines), companion animals (e.g., canines, felines) and rodents (e.g., mice, rabbits and rats).

“Treating” a disease in a subject or “treating” a subject having a disease refers to subjecting the subject to a pharmaceutical treatment, e.g., the administration of a drug, such that at least one symptom of the disease is decreased or prevented from worsening.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

Unless otherwise defined herein, scientific and technical terms used in this application shall have the meanings that are commonly understood by those of ordinary skill in the art. Generally, nomenclature and techniques relating to chemistry, molecular biology, cell and cancer biology, immunology, microbiology, pharmacology, and protein and nucleic acid chemistry, described herein, are those well-known and commonly used in the art.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual and partial numbers within that range, for example, 1, 2, 3, 4, 5, 5.5 and 6. This applies regardless of the breadth of the range.

### Platelet Binding Proteins (PBPs)

### Fibrinogen Mimetic Peptides (FMPs)

As used herein, the terms “fibrinogen mimetic peptide” and the term “active platelet GPIIb-IIIa-binding peptide” are used interchangeably in the present disclosure. In some aspects, provided herein is a fibrinogen mimetic peptide of Formula (I):
cyclo-(CNPRGD{Tyr(OEt)}R-β-A) (SEQ ID NO: 1), referred to herein as “FMP2”, or a salt thereof.

In some embodiments, the fibrinogen mimetic peptide is FMP1, which has the formula: cyclo-(CNPRGD{Tyr(OEt)}R-β-C) (SEQ ID NO: 2), or a salt thereof.

In some embodiments, the FMP is linear RGD (GRGDSP (SEQ ID NO: 3)).

In some embodiments, the FMP is H12 (HHLGGAKQAGDV (SEQ ID NO: 4)).

In some embodiments, the salt of the fibrinogen mimetic is an acetate salt, or a trifluoroacetate salt. In some embodiments, the fibrinogen mimetic peptide specifically binds to activated GPIIb-IIIa. In some embodiments, the fibrinogen mimetic peptide inhibits platelet aggregation. In some embodiments, the fibrinogen mimetic peptide has an IC₅₀ less than 30 μM, e.g., less than 25 μM, less than 20 μM, less than 15 μM, less than 10 μM, less than 5 μM, less than 1 μM, less than 0.5 μM, less than 0.1 μM, less than 0.05 μM, less than 4.5×10⁻² μM, less than 4.0×10⁻² M, less than 3.5×10⁻² M, less than 3.0×10⁻² μM, less than 2.5×10⁻² M, less than 2.0×10⁻² M, less than 1.5×10⁻² M, less than 1.0×10⁻² μM, less than 0.5×10⁻² M, less than 0.1×10⁻² μM, less than 0.5×10⁻³ μM, less than 0.1×10⁻³ μM, etc. In certain embodiments, the fibrinogen mimetic peptide has an IC₅₀ of about 0.13 μM.

In some embodiments, the fibrinogen mimetic peptide disclosed herein is conjugated to a polymer (e.g., a lipid, a protein, etc.). Therefore, in some embodiments, provided herein are fibrinogen mimetic peptide conjugates comprising a fibrinogen mimetic peptide of SEQ ID NO: 1, 2, 3, or 4 conjugated to a polymer. In some embodiments, the polymer is a lipid (e.g., DSPE-PEG(2k)-maleimide). In some embodiments, the fibrinogen mimetic peptide is conjugated to the polymer through thio-ene coupling to the thiol group on an existing or added N-terminal cysteine or 3-mercaptopropionic acid. In some embodiments, the surface of a particle (e.g., a synthetic platelet) comprises an FMP peptide, or a salt thereof.

In some embodiments, the FMP can include an RGD amino acid sequence motif that promotes active platelet aggregation. The RGD motif containing FMP may contain a single repeat of the RGD motif or may contain multiple repeats of the RGD motif, such as, for example, 2, 3, 4, 5, 6, 7, 8, 9, 10, or more repeats of the RGD motif. One of skill in the art will understand that conservative substitutions of particular amino acid residues of the RGD motif containing FMPs may be used so long as the RGD motif containing FMP retains the ability to bind comparably as the native RGD motif. One of skill in the art will also understand that conservative substitutions of particular amino acid residues flanking the RGD motif so long as the RGD motif containing FMP retains the ability to bind comparably to the native RGD motif.

In some embodiments, the FMP can be a fibrinogen mimetic peptide (FMP) described herein. In some embodiments, FMP is of Formula (I). A cyclic peptide of Formula (I) can have high selectivity and affinity to GPIIb-IIIa on activated platelets but do not bind or activate quiescent platelets nor interact with other RGD-binding integrins. The FMP can be synthesized using Fmoc-based solid phase chemistry on Knorr resin and characterized using mass spectroscopy.

Advantageously, the FMPs can each include about 5 to about 30 amino acids. By limiting the size of the peptides to about 5 to about 30 amino acids, the FMPs can be spatially or topographically arranged on the flexible particle surface such that the FMPs do not spatially mask each other and are able to promote arrest and aggregation of active platelets onto injury sites.

### P-Selectin Binding Peptide

As used herein, the term “P-selectin binding peptide” refers to a protein or peptide that binds to P-selectin on platelets with high affinity (nano-micromolar affinity), such as glycosulfopeptide mimics of the N-terminal of PSGL-1 (native ligand for P-selectin) or peptides derived from phage display such as EWVDV-containing peptides. In certain embodiments, the P-selectin binding peptide has the amino acid sequence DAEWVDVS (SEQ ID NO: 5).

In some embodiments, the P-selectin binding peptide disclosed herein is conjugated to a polymer (e.g., a lipid, a protein, etc.). Therefore, in some embodiments, provided herein are P-selectin binding peptide conjugates comprising a P-selectin binding peptide of SEQ ID NO: 5 conjugated to a polymer. In some embodiments, the polymer is a lipid (e.g., DSPE-PEG(2k)-maleimide). In some embodiments, the P-selectin binding peptide is conjugated to the polymer through thio-ene coupling to the thiol group on an added N-terminal cysteine or 3-mercaptopropionic acid. In some embodiments, the surface of a particle (e.g., a synthetic platelet) comprises a P-selectin binding peptide, or a salt thereof.

In some embodiments, the P-selectin binding peptide can be a peptide described herein. In specific embodiments, the P-selectin binding peptide having the amino acid sequence of DAEWVDVS (SEQ ID NO: 5). A peptide having the amino acid sequence of SEQ ID NO: 5 can have high selectivity and affinity to P-selectin on activated platelets. The P-selectin binding peptide can be synthesized using FMoc-based solid phase chemistry on Knorr resin and characterized using mass spectroscopy.

Advantageously, the P-selectin binding peptide can each include about 5 to about 30 amino acids. By limiting the size of the peptides to about 5 to about 30 amino acids, the P-selectin binding peptides can be spatially or topographically arranged on the flexible particle surface such that they do not spatially mask each other and are able to promote arrest and aggregation of active platelets onto injury sites.

### Von Willebrand Binding Peptide (VBP)

As used herein, the term “von Willebrand binding peptide” refers to a protein or peptide that binds to von Willebrand Factor with high affinity (nano-micromolar affinity). Von Willebrand Factor has multiple binding domains, so the VBP could consist of a peptide that binds to the D′D3 domain (such as Factor FVIII-derived peptides), the A1 or A3 domains (such as collagen-derived/mimetic peptides), or A1 or C4 domains (such as platelet GPIb or GPIIb-IIIa-derived peptides). In certain embodiments, the VBP has the amino acid sequence

(SEQ ID NO: 6)
TRYLRIHPQSWVHQI.

In some embodiments, the VBP disclosed herein is conjugated to a polymer (e.g., a lipid, a protein, etc.). Therefore, in some embodiments, provided herein are VBP conjugates comprising a VBP of SEQ ID NO: 6 conjugated to a polymer. In some embodiments, the polymer is a lipid (e.g., DSPE-PEG(2k)-maleimide). In some embodiments, the VBP is conjugated to the polymer through thio-ene coupling to the thiol group on an added N-terminal cysteine or 3-mercaptopropionic acid. In some embodiments, the surface of a particle (e.g., a synthetic platelet) comprises a VBP, or a salt thereof.

In some embodiments, the VBP peptide for vWF binding can include a recombinant GPIbα fragment (rGPIbα) containing the vWF binding sites (residues 1 to 302) or a short chain vWF-binding peptide. The GPIbα fragment can be expressed in CHO cells and isolated, adapting methods described. The short VBP can comprise the amino acid sequence TRYLRIHPQSWVHQI (SEQ ID NO: 6), A peptide having an amino acid sequence of SEQ ID NO: 6 can be synthesized using fluorenylmethyloxycarbonyl chloride (FMoc)-based solid phase chemistry on Knorr resin, and characterized using mass spectroscopy. Each vWF molecule has only one binding region for this peptide, and hence vascular injury sites presenting multiple vWF binding sites for multiple copies of this peptide decorated on the particle surface provide a mechanism for enhanced adhesion of the particles with increasing shear.

### Collagen Binding Peptide (CBP)

As used herein, the term “collagen binding peptide” refers to a protein or peptide that binds to collagen with high affinity (nano-micromolar affinity), such as collagen-derived sequences (such as GPO repeats) that have hellicogenic affinity for collagen or collagen binding peptides derived experimentatlly (such as phage display or isothermal titration chemistry). In certain embodiments, the CBP has the amino acid sequence (GPO)₇ (SEQ ID NO: 7).

In some embodiments, the CBP disclosed herein is conjugated to a polymer (e.g., a lipid, a protein, etc.). Therefore, in some embodiments, provided herein are CBP conjugates comprising a CBP of SEQ ID NO: 7 conjugated to a polymer. In some embodiments, the polymer is a lipid (e.g., DSPE-PEG(2k)-maleimide). In some embodiments, the CBP is conjugated to the polymer through thio-ene coupling to the thiol group on an added N-terminal cysteine or 3-mercaptopropionic acid. In some embodiments, the surface of a particle (e.g., a synthetic platelet) comprises a CBP, or a salt thereof.

In some embodiments, the CBP can comprise a peptide that comprises a short repeat of the tripeptide GPO, such as (GPO)₇ SEQ ID NO: 7, with a helicogenic affinity to fibrillar collagen. The GPO trimer is based on amino acid repeats found in the native collagen structure.

It has been reported that the activation of platelets usually caused by interaction with collagen through GPVI and GPIa/IIa, can also potentially occur when platelets interact with collagen-derived peptides. This can be a potential problem regarding decorating synthetic particle surfaces with collagen-derived peptides for binding of collagen because in vivo the constructs can potentially interact with quiescent blood platelets and systemically activate them, posing thromboembolic risks. However, interaction of platelet receptors with collagen and the subsequent platelet activation mechanisms are dependent upon receptor clustering induced by multimeric long chain triple-helical fibrillar collagen and not by short collagen-mimetic peptide repeats. In fact, it has been shown that GPO-trimer repeats as high as a 30-mer (10 repeats) only partially interact with platelet GPIa/IIa and GPVI integrins and are incapable of activating platelets; yet they can effectively bind to fibrillar collagen via helicogenic interaction. Hence, this small CBP can promote adhesion to fibrillar collagen, but cannot activate quiescent platelets due to absence of long triple-helical conformation. The CBP like the VBP can also be synthesized using FMoc-based solid phase chemistry on Knorr resin and characterized using mass spectroscopy.

### Synthetic Platelets

In some aspects, the present disclosure relates to particles that function as synthetic platelets. In certain embodiments, the particles are conjugated to a plurality of Platelet Binding Peptides (PBPs), such as FMP1 peptides, FMP2 peptides, linear RGD peptides, H12 peptides, and/or P-selectin binding peptides; CBPs; and VBPs described herein. For example, in some embodiments, the particle is conjugated to an FMP1 peptide. In some embodiments, the particle is conjugated to an FMP2 peptide. In some embodiments, the particle is conjugated to a linear RGD peptide. In some embodiments, the particle is conjugated to an H12 peptide. In some embodiments, the particle is conjugated to a P-selectin binding peptide. In some embodiments, the particle is conjugated to a CBP. In some embodiments, the particle is conjugated to a VBP. Methods of using these particles in diminishing bleeding and blood loss are provided, as well as compositions and methods useful in the delivery of therapeutic agents to the vasculature. The synthetic platelets described herein integrate platelet-mimetic adhesion- and aggregation-promoting functionalities on a single flexible particle. It was found that the platelet-mimetic adhesion- and aggregation-promoting functionalities can be achieved by including on, conjugating to, or decorating a flexible particle with a plurality of three peptides, i.e., a VBP, a CBP, and a PBP. It was initially found that liposomes bearing VBP and CBP motifs undergo platelet-mimetic adhesion under flow on vWF and collagen-coated surfaces in vitro at low-to-high shear in parallel plate flow chamber (PPFC) experiments and that PBP-modified liposomes pre-adhered to a surface can enhance the aggregation of ADP-activated platelets onto them, even at low platelet concentrations. Subsequently, it was found that liposomes bearing all three peptides (VBP, CBP and PBP), when introduced in PPFC flow along with low concentration of ADP-activated platelets over a vWF/collagen mixed coated surface, are able to adhere to the surface under high shear and promote arrest and aggregation of active platelets onto sites of liposome adhesion. In some embodiments, the PBP included in the synthetic platelets is a fibrinogen mimetic peptide (FMP) described herein. In some embodiments, the FMP is FMP1, FMP2, linear RGD, or H12, or a combination thereof. In some embodiments, the PBP included in the synthetic platelets is a P-selectin binding peptide described herein.

It is therefore an aspect of the application that administration, such as intravenous administration, of the synthetic platelets described herein to a subject with a vascular injury can diminish the bleeding time in the subject. It is a further aspect of the application that the synthetic platelets provide a nanostructure that binds with a vascular injury site as well as activated platelets and enhances their rate of adhesion and aggregation to aid in stopping bleeding.

In some embodiments, the synthetic platelets described herein can include a biocompatible, biodegradable, flexible particle core and a plurality of VBPs, CBPs, and PBPs bound to, conjugated to, and/or decorated on the a surface defined by the flexible particle core. The VBPs, CBPs, and PBPs can be spatially or topographically arranged on the flexible particle surface such that the VBPs, CBPs, and PBPs do not spatially mask each other and are able to adhere to a vascular surface, vascular disease site, and/or vascular injury site with exposed vWF and collagen and promote arrest and aggregation of active platelets onto sites of particle adhesion.

The biocompatible, biodegradable, flexible particles be made from any biocompatible, biodegradable material that can form a flexible particle to which the peptides described herein can be attached, conjugated, and/or decorated. In some embodiments, the biocompatible, biodegradable flexible particles can include a liposome, a hydrogel, micelle, and/or polymer, which can include and/or be surface modified or engineered with the VBPs, CBPs, and PBPs.

The liposome or hydrogel can include a lipid and/or any naturally occurring, synthetic or semi-synthetic (i.e., modified natural) moiety that is generally amphipathic (i.e., including a hydrophilic component and a hydrophobic component). Examples of lipids can include fatty acids, neutral fats, phospholipids, oils, glycolipids, surfactants, aliphatic alcohols, waxes, terpenes and steroids. Semi-synthetic or modified natural lipids can include natural lipids that have been chemically modified in some fashion. The at least one lipid can be neutral, negatively charged (i.e., anionic), or positively charged (i.e., cationic). Examples of anionic lipids can include phosphatidic acid, phosphatidyl glycerol, and fatty acid esters thereof, amides of phosphatidyl ethanolamine, such as anandamides and methanandamides, phosphatidyl serine, phosphatidyl inositol and fatty acid esters thereof, cardiolipin, phosphatidyl ethylene glycol, acidic lysolipids, sulfolipids and sulfatides, free fatty acids, both saturated and unsaturated, and negatively charged derivatives thereof. Examples of cationic lipids can include N-[1-(2,3-dioleoyloxy)propyl]-N,N,N-trimethyl-ammonium chloride and common natural lipids derivatized to contain one or more basic functional groups.

Other examples of lipids, any one or combination of which may be used to form the particle, can include: phosphocholines, such as 1-alkyl-2-acetoyl-sn-glycero 3-phosphocholines, and 1-alkyl-2-hydroxy-sn-glycero 3-phosphocholines; phosphatidylcholine with both saturated and unsaturated lipids, including dioleoylphosphatidylcholine, dimyristoylphosphatidylcholine, dipentadecanoylphosphatidylcholine, dilauroylphosphatidylcholine, dipalmitoylphosphatidylcholine (DPPC), distearoylphosphatidylcholine (DSPC), and diarachidonylphosphatidylcholine (DAPC); phosphatidylethanolamines, such as dioleoylphosphatidylethanolamine, dipalmitoylphosphatidylethanolamine (DPPE), and distearoylphosphatidylethanolamine (DSPE); phosphatidylserine; phosphatidylglycerols, including distearoylphosphatidylglycerol (DSPG); phosphatidylinositol; sphingolipids, such as sphingomyelin; glycolipids, such as ganglioside GM1 and GM2; glucolipids; sulfatides; glycosphingolipids; phosphatidic acids, such as dipalmitoylphosphatidic acid (DPPA) and distearoylphosphatidic acid (DSPA); palmitic acid; stearic acid; arachidonic acid; oleic acid; lipids bearing polymers, such as chitin, hyaluronic acid, polyvinylpyrrolidone or polyethylene glycol (PEG); lipids bearing sulfonated mono-, di-, oligo- or polysaccharides; cholesterol, cholesterol sulfate, and cholesterol hemisuccinate; tocopherol hemisuccinate; lipids with ether and ester-linked fatty acids; polymerized lipids (a wide variety of which are well known in the art); diacetyl phosphate; dicetyl phosphate; stearylamine; cardiolipin; phospholipids with short chain fatty acids of about 6 to about 8 carbons in length; synthetic phospholipids with asymmetric acyl chains, such as, for example, one acyl chain of about 6 carbons and another acyl chain of about 12 carbons; ceramides; non-ionic liposomes including niosomes, such as polyoxyalkylene (e.g., polyoxyethylene) fatty acid esters, polyoxyalkylene (e.g., polyoxyethylene) fatty alcohols, polyoxyalkylene (e.g., polyoxyethylene) fatty alcohol ethers, polyoxyalkylene (e.g., polyoxyethylene) sorbitan fatty acid esters (such as, for example, the class of compounds referred to as TWEEN (commercially available from ICI Americas, Inc., Wilmington, Del.), glycerol polyethylene glycol oxystearate, glycerol polyethylene glycol ricinoleate, alkyloxylated (e.g., ethoxylated) soybean sterols, alkyloxylated (e.g., ethoxylated) castor oil, polyoxyethylene-polyoxypropylene polymers, and polyoxyalkylene (e.g., polyoxyethylene) fatty acid stearates; sterol aliphatic acid esters including cholesterol sulfate, cholesterol butyrate, cholesterol isobutyrate, cholesterol palmitate, cholesterol stearate, lanosterol acetate, ergosterol palmitate, and phytosterol n-butyrate; sterol esters of sugar acids including cholesterol glucuronide, lanosterol glucuronide, 7-dehydrocholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl gluconate; esters of sugars and aliphatic acids including sucrose laurate, fructose laurate, sucrose palmitate, sucrose stearate, glucuronic acid, gluconic acid and polyuronic acid; saponins including sarsasapogenin, smilagenin, hederagenin, oleanolic acid, and digitoxigenin; glycerol dilaurate, glycerol trilaurate, glycerol dipalmitate, glycerol and glycerol esters including glycerol tripalmitate, glycerol distearate, glycerol tristearate, glycerol dimyristate, glycerol trimyristate; long chain alcohols including n-decyl alcohol, lauryl alcohol, myristyl alcohol, cetyl alcohol, and n-octadecyl alcohol; 6-(5-cholesten-3-yloxy)-1-thio-D-galactopyranoside; digalactosyldiglyceride; 6-(5-cholesten-3-yloxy)hexyl-6-amino-6-deoxy-1-thio-D-galactopyranoside; 6-(5-cholesten-3-yloxy)hexyl-6-amino-6-deoxyl-1-thio-a-D-mannopyranoside; 12-(((7′-diethylaminocoumarin-3-yl)carbonyl)methylamino)octadecanoic acid; N-[12-(((7′-diethylaminocoumarin-3-yl)carbonyl)methylamino)octadecanoyl]-2-aminopalmitic acid; cholesteryl(4′-trimethylammonio)butanoate; N-succinyldioleoylphosphatidylethanolamine; 1,2-dioleoyl-sn-glycerol; 1,2-dipalmitoyl-sn-3-succinylglycerol; 1,3-dipalmitoyl-2-succinylglycerol; 1-hexadecyl-2-palmitoylglycerophosphoethanolamine and palmitoylhomocysteine; and/or any combinations thereof.

Examples of biocompatible, biodegradable polymers that can be used to form the particles are poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and poly(lactide)s or poly(lactide-co-glycolide)s, biodegradable polyurethanes, and blends and/or copolymers thereof.

Other examples of materials that may be used to form the particles can include chitosan, poly(ethylene oxide), poly(lactic acid), poly(acrylic acid), poly(vinyl alcohol), poly(urethane), poly(N-isopropyl acrylamide), poly(vinyl pyrrolidone) (PVP), poly(methacrylic acid), poly(p-styrene carboxylic acid), poly(p-styrenesulfonic acid), poly(vinylsulfonicacid), poly(ethyleneimine), poly(vinylamine), poly(anhydride), poly(L-lysine), poly(L-glutamic acid), poly(gamma-glutamic acid), poly(carprolactone), polylactide, poly(ethylene), poly(propylene), poly(glycolide), poly(lactide-co-glycolide), poly(amide), poly(hydroxylacid), poly(sulfone), poly(amine), poly(saccharide), poly(HEMA), poly(anhydride), gelatin, glycosaminoglycans (GAG), poly(hyaluronic acid), poly(sodium alginate), alginate, albumin, hyaluronan, agarose, polyhydroxybutyrate (PHB), copolymers thereof, and blends thereof.

The flexible particles can have a maximum length or diameter of about 100 nm to about m and a substantially spherical, discoidal, and/or ellipsoidal shape. The physical size and shape as well as mechanical properties of the particles can be engineered to mimic those of natural platelets that are important in hemostasis. In some embodiments, the flexible particles can have an about 2 to about 5 μm diameter discoidal shape and an about 10 to about 50 kPa mechanical elastic modulus that mimics the size, shape, and elastic modulus of platelets and facilitates upon administration to the vasculature of a subject their margination to the vascular wall and their bio-interactions.

In an embodiment of the application, oblate ellipsoid particles having a diameter of about 2 to about 5 μm and a mechanical modulus of about 10 to about 50 kPa can be prepared by initially forming a polymer template. The polymer template can then be used to build a protein/polymer shell using a cross-linked layer-by-layer assembly. The polymer template can subsequently be removed using solvents to leave behind soft, flexible, proteinaceous discoid particles having a diameter about 2 to about 5 μm and a mechanical elastic modulus of about 10 to about 50 kPa. The particles can then be surface-modified with the VBPs, CBPs, and PBPs at a surface density effective to promote maximum particle adhesion to vWF and collagen exposed surfaces at low-to-high sheer stresses and promote aggregation of active platelets even at low (less about 50,000 per 1) platelet concentrations.

By way of example, poly-1-lactide-co-glycolide (PLGA) spherical particles having a diameter of about 2 to about 3 μm can be embedded into polyvinyl alcohol (PVA) film (e.g., about 5% w/v in water) containing 2% (v/v) glycerol as a plasticizer and biaxially stretched to twice the original length and width in an oven at about 65° C. The film can be removed from the stretcher and the PVA dissolved in 15% isopropanol followed by thorough washing with isopropanol to ensure complete removal of PVA. This results in the recovery of the oblate PLGA particles that can be resuspended in distilled water or PBS. These template particles can then be coated with protein and polyelectrolyte layers using a layer-by-layer (LBL) technique. For this, protein serum albumin (SA, e.g., human serum albumin or mouse serum albumin) and the polyelectrolyte polyallylamine hydrochloride (PAH) can be used at a 2 mg/ml concentration for adsorption. At the pH employed, albumin is negatively charged and PAH is cationic, alternate layers of SA and PAH can be formed on the PLGA template particles via electrostatic interactions. Multiple alternating layers (e.g., at least seven layers) can be formed on the oblate template and cross-linked with gluteraldehyde intermittently to enhance stability. The particles can then be exposed to a solvent mixture (e.g., 2:1 tetrahydrofuran:isopropanol) to dissolve the PLGA core, leaving behind the LBL deposited soft SA/PAH flexible discoid shell. The outermost layer can include albumin so that PEGylated peptides describe herein can be readily attached.

The VBPs, CBPs, and PBPs can be conjugated to the particle surface by reacting the peptides through a thiol group on an existing or added N-terminal cysteine or 3-mercaptopropionic acid to a maleimide-terminated lipid, such as maleimide-PEG-DSPE. The lipid-peptide conjugates can then be incorporated into lipophillic particles such as liposomes using known formulation techniques.

The VBPs, CBPs, and PBPs can be conjugated to the particle surface by reacting the peptides with through their N-termini to the carboxyl termini of a heterobifunctional PEG, such as maleimide-PEG-COOH. The PEG-peptide conjugates or PEGylated peptides can then be conjugated to the particle using known conjugation techniques.

The PEG molecules can have a variety of lengths and molecular weights, including, for example, PEG 200, PEG 1000, PEG 1500, PEG 2000, PEG 4600, PEG 10,000, or combinations thereof. In other embodiments, the VBPs, CBPs, and PBPs can be conjugated to the particle surface with PEG acrylate, or PEG diacrylate, molecules of a variety of molecular weights.

In one example, the VBPs, CBPs, and PBPs can be reacted with maleimide-PEG-COOH to form Mal-PEG-peptide conjugates. SA/PAH particles with albumin as the outermost layer can then be treated with dithiothreitol (DTT) to introduce a high density of sulfhydryl (—SH) groups on the surface. The Mal-PEG-peptides can then be incubated with the DTT-treated particles, such that the MAL termini can react with the free SH groups to form particles decorated with various peptides presented on the particle surface via PEG linkers.

The relative amounts of the peptides conjugated to the particle surface can affect the efficiency of the particle's hemostatic activity. In some embodiments, the molar percentage of the PBP, CBP, and/or VBP conjugated to the particle's surface is <5 molar percent. In some embodiments the molar percentage of the PBP, CBP, and/or VBP is between 5% and about 0.5%, between 5% and about 1%, between 5% and about 2%, between 5% and about 3%, between 5% and about 4%, exclusive of 5% and inclusive of the lower range limit. In some embodiments the molar percentage of the PBP, CBP, and/or VBP is between about 4% and about 0.1%, between about 3% and about 0.1%, between about 2% and about 0.1%, between about 1% and about 0.1%, between about 4% and about 0.5%, between about 3% and about 0.5%, between about 2% and about 0.5%, or between about 1% and about 0.5%.

The ratio of VBPs to CBPs conjugated on the particle surface can be about 70:30 to about 30:70 and be adjusted accordingly to maximize adhesion under low-to-high shear conditions. In some embodiments, the ratio of VBP:CPB:PBP can be about 1:1:2 to 1:2:1 to 2:1:1. In some embodiments, the relative molar ratios of PBP:CBP:VBP are 1:5:5. In some embodiments, the relative molar ratios are between about 1:1:1 and about 1:5:1, between about 1:1:1 and about 1:1:5, between about: 1:1:1 and about 1:5:5, between about 1:1:1 and about 5:1:1, between about 1:1:1 and about 5:5:1, or between about 1:1:1 and about 5:1:5. In some embodiments, the relative molar ratios of PBP:CBP:VBP are about 2:1:1, about 1:5:5, about 10:5:1, about 10:1:5, about 1:2:1, about 1:1:2, about 10:1:1, about 2:1:0, about 2:0:1, about 1:0:0, about 0:1:0, about 0:0:1, or about 0:1:1, or any ratio between any two of these ratios. It will be appreciated, that other ratios can be used to enhance the particle adherence and activated platelet aggregation.

In some embodiments, the compositions comprising a synthetic platelet described herein, can be formulated and administered to an animal, preferably a human, in need of reducing or slowing blood loss. In other embodiments, the compositions comprising a synthetic platelet described herein, may be formulated and administered to an animal, preferably a human, to facilitate the delivery of a therapeutic agent.

In some embodiments, the synthetic platelets described herein can be provided in a pharmaceutical composition. Such a pharmaceutical composition may consist of a synthetic platelet alone, in a form suitable for administration to a subject, or the pharmaceutical composition may comprise a synthetic platelet and one or more pharmaceutically acceptable carriers, one or more additional ingredients, one or more pharmaceutically acceptable therapeutic agents, bioactive agents, diagnostic agents, or some combination of these. The therapeutic agent may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the therapeutic agent may be combined and which, following the combination, can be used to administer the therapeutic agent to a subject.

As used herein, the term “physiologically acceptable” ester or salt means an ester or salt form of the therapeutic agent which is compatible with any other ingredients of the pharmaceutical composition, which is not deleterious to the subject to which the composition is to be administered.

In some embodiments, the bioactive agent, diagnostic agent, and/or therapeutic agent can be conjugated, encapsulated, and/or contained with the synthetic platelet so that synthetic platelet acts as a delivery vehicle. In other embodiments, the bioactive agent, diagnostic agent, and/or therapeutic agent can be merely contained in a pharmaceutical composition either with or without the synthetic platelets and administered to concurrently with or separately from administration of the synthetic platelets. Selection of a bioactive agent, diagnostic agent, and/or therapeutic agent to be conjugated to or encapsulated within the synthetic platelet is dependent upon the use of the synthetic platelet and/or the condition being treated and the site and route of administration.

Bioactive agents encapsulated by and/or conjugated to the synthetic platelet can include any substance capable of exerting a biological effect in vitro and/or in vivo. Examples of bioactive agents can include, but are not limited to, biologically active ligands, small molecules, proteins, DNA fragments, DNA plasmids, interfering RNA molecules, such as siRNAs, mRNAs, oligonucleotides, and DNA encoding for shRNA. Diagnostic agents can include any substance that may be used for imaging a region of interest (ROI) in a subject and/or diagnosing the presence or absence of a disease or diseased tissue in a subject. Therapeutic agents can refer to any therapeutic or prophylactic agent used in the treatment (including the prevention, diagnosis, alleviation, or cure) of a malady, affliction, condition, disease or injury in a subject. It will be appreciated that the membrane can additionally or optionally include proteins, carbohydrates, polymers, surfactants, and/or other membrane stabilizing materials, any one or combination of which may be natural, synthetic, or semisynthetic.

The methods of treatment using the synthetic platelets described herein include administering a therapeutically effective amount of a synthetic platelet to a subject in need thereof. It should be understood, that the methods of treatment by the delivery of a synthetic platelet include the treatment of subjects that are already bleeding, as well as prophylactic treatment uses in subjects not yet bleeding. In a preferred embodiment, the subject is an animal. In a more preferred embodiment, the subject is a human.

In some aspects, methods of treating a subject having or suspected of having cancer are provided in which the subject is administered a pharmaceutical composition comprising a particle described herein. In some embodiments, the pharmaceutical composition comprises the particle and an anti-cancer therapeutic agent. In some embodiments, the particle encapsulates or is conjugated to the anticancer agent.

In some aspects, methods are provided for preventing or inhibiting platelet aggregation in a subject in need thereof, the method comprising administering to the subject a pharmaceutical composition comprising a fibrinogenic mimetic peptide described herein The embodiments described herein should in no way be construed to be limited to the synthetic platelets described herein, but rather should be construed to encompass the use of additional synthetic platelets, both known and unknown, that diminish or reduce bleeding or blood loss.

The formulations of the pharmaceutical compositions described herein may be prepared by any method known or hereafter developed in the art of pharmacology. In general, such preparatory methods include the step of bringing a synthetic platelet into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions, which are suitable for ethical administration to humans, it will be understood by the skilled artisan that such compositions are generally for administration to animals of all sorts. Modification of pharmaceutical compositions for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, animals including commercially relevant animals such as cattle, pigs, horses, sheep, cats, and dogs, birds including commercially relevant birds such as chickens, ducks, geese, and turkeys.

Pharmaceutical compositions that are useful in the methods described herein may be administered, prepared, packaged, and/or sold in formulations for parenteral, oral, rectal, vaginal, topical, transdermal, pulmonary, intranasal, buccal, ophthalmic, or another route of administration.

The compositions described herein may be administered via numerous routes, including, but not limited to, parenteral, oral, rectal, vaginal, topical, transdermal, pulmonary, intranasal, buccal, or ophthalmic administration routes. The route(s) of administration will be readily apparent to the skilled artisan and will depend upon any number of factors including the type and severity of the disorder being treated, the type and age of the veterinary or human patient being treated, and the like.

Parenteral administration of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition on or through a surgical incision, by application of the composition on or through a tissue-penetrating non-surgical wound, and the like. In particular, parenteral administration is contemplated to include, but is not limited to, cutaneous, subcutaneous, intraperitoneal, intramuscular, intrasternal, intravenous, and intra-arterial administration.

Formulations of a pharmaceutical composition suitable for parenteral administration comprise the therapeutic agent combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles. Such formulations may further comprise one or more additional ingredients including, but not limited to, suspending, stabilizing, or dispersing agents. In one embodiment of a formulation for parenteral administration, the therapeutic agent is provided in dry (i.e., powder or granular) form for reconstitution with a suitable vehicle (e.g., sterile pyrogen free water) prior to parenteral administration of the reconstituted composition.

Pharmaceutical compositions that are useful in the methods described herein may be administered systemically in oral solid formulations, ophthalmic, suppository, aerosol, topical or other similar formulations. In addition to the compound such as heparin sulfate, or a biological equivalent thereof, such pharmaceutical compositions may contain pharmaceutically acceptable carriers and other ingredients known to enhance and facilitate administration.

The pharmaceutical compositions described herein may also be formulated so as to provide slow, prolonged or controlled release. In general, a controlled-release preparation is a pharmaceutical composition capable of releasing the synthetic platelet at a desired or required rate to maintain constant activity for a desired or required period of time.

A pharmaceutical composition described herein may be prepared, packaged, or sold in bulk, as a single unit dose, or as a plurality of single unit doses. As used herein, a “unit dose” is discrete amount of the pharmaceutical composition comprising a predetermined amount of the activity. The amount of the activity is generally equal to the dosage, which would be administered to a subject or a convenient fraction of such a dosage such as, for example, one-half or one-third of such a dosage.

The relative amounts of the ingredients in a pharmaceutical composition of the invention will vary, depending upon the identity, size, and condition of the subject treated and further depending upon the route by which the composition is to be administered. By way of a non-limiting example, the composition may comprise between 0.1% and 100% (w/w) of the synthetic platelets.

The synthetic platelet compositions described herein may be administered to deliver a dose of between about 1 ng/kg/day and about 100 mg/kg/day. In one embodiment, a dose can be administered that results in a concentration of the synthetic platelets between about 0.01 g/mL and about 625 μg/mL in the blood of a mammal. While the precise dosage administered will vary depending upon any number of factors, including but not limited to, the type of animal, the amount of bleeding being treated, the type of bleeding being treated, the type of wound being treated, the age of the animal and the route of administration. Preferably, the dosage of the synthetic platelet will vary from about 1 μg to about 50 mg per kilogram of body weight of the animal. More preferably, the dosage will vary from about 10 g to about 15 mg per kilogram of body weight of the animal. Even more preferably, the dosage will vary from about 100 μg to about 10 mg per kilogram of weight of the animal.

The pharmaceutical compositions may be prepared, packaged, or sold in the form of a sterile injectable aqueous or oily suspension or solution. This suspension or solution may be formulated according to the known art, and may comprise, in addition to the therapeutic agent, additional ingredients such as the dispersing agents, wetting agents, or suspending agents described herein. Such sterile injectable formulations may be prepared using a non-toxic parenterally acceptable diluent or solvent, such as water or 1,3butane diol, for example. Other acceptable diluents and solvents include, but are not limited to, Ringer's solution, isotonic sodium chloride solution, and fixed oils such as synthetic mono or di-glycerides.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The pharmaceutical composition may be administered to an animal as needed. The pharmaceutical composition may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### EXAMPLES

### Example 1: Technical Study Summary

### A. Study Purpose and Background

The purpose of this study was to compare the effectiveness of a new fibrinogen mimetic peptide (FMP) peptide sequence against a previously used sequence. The original FMP peptide sequence is cyclo-{Pra}CNPRGD{Tyr(OEt)}RC trifluoroacetate salt. The new peptide sequence is cyclo-(CNPRGD{Tyr(OEt)}R-β-A) trifluoroacetate salt.

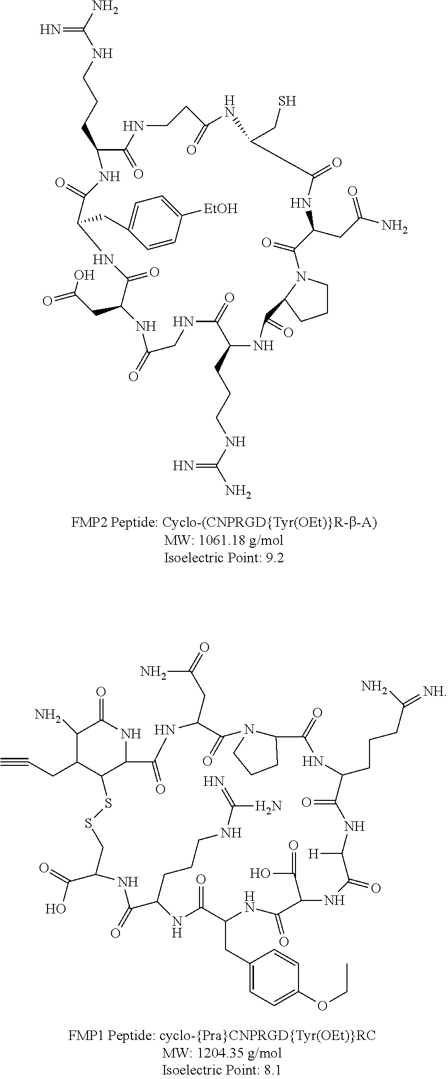

### B. Methods

The properties of the FMP2 peptide and peptide-decorated particles (synthetic platelet) were compared to those of the old FMP1 peptide. First, Chang et al. [1] have shown that cyclic RGD peptides are able to selectively bind to activated platelets and prevent human platelet aggregation as measured via platelet light transmission aggregometry. This protocol was adapted and performed to compare the IC₅₀ values of FMP1 and FMP2. Specifically, human whole blood was drawn from healthy, aspirin refraining donors via venipuncture into syringe containing 3.8% w/v sodium citrate. Platelet rich plasma (PRP) was obtained by centrifugation of whole blood in a tabletop centrifuge at 150×g for 15 minutes. Platelet aggregation experiments were performed with 225 μl of 25% o (v/v) diluted PRP stimulated with 25 μl of 10 μM adenosine 5′-diphosphate (ADP) (or saline as a negative control) on a Bio/Data PAP-8E Platelet Aggregation Profiler. 20 μl of saline containing 0.0001 μM to 250 μM of FMP1 or FMP2 (at each order of magnitude) were added to the 25% o diluted PRP, and final aggregation percentage was recorded. In GraphPad Prism®, a non-linear fit of log-dose vs. response was applied to the data, and the IC₅₀ for each peptide was determined by calculating the intersection point at y=50. The acceptance criteria for this assay was to show that FMP2 IC₅₀≤FMP1 IC₅₀.

Next, signs of dimer/trimerization in peptides, which is an indicator of poor stability, was investigated using matrix-assisted laser desorption ionization time of flight mass spectroscopy (MALDI-TOF) analysis using a Bruker UltraFlex III MALDI-TOF/TOF mass spectrometer (S/N 247420 00194). The samples were prepared using a sandwich matrix method with 5 μL of the matrix trans-2-[3-(4-Tert-Butylphenyl)-2-methyl-2-propenylidene]malononitrile (DCTB, 25 mg/mL) sandwiching 5 μL of the samples at 20 mg/mL in methanol, The samples were measured using both Positive Reflectron Mode (1000-10,000 m/z range) and Positive Linear Mode (500-5000 m/z range).

FMP peptides were then conjugated to functionalized lipid-PEG molecules to generate lipopeptides. FMP1 was conjugated to DSPE-PEG(2000)-azide via the alkyne moiety of propargylglycine using copper-catalyzed azide-alkyne click chemistry, and the conjugation was confirmed using MALDI-TOF. FMP2 was conjugated to DSPE-PEG(2000)-maleimide via the sulfhydryl moiety to form a stable thioether linkage, and the conjugation was confirmed using MALDI-TOF.

These lipid-peptide conjugates were formulated into synthetic platelet particles (Table 1) using standard lipid film rehydration and extrusion methods. Briefly, DSPC, cholesterol, DSPE-PEG(2000)-FMP1 or DSPE-PEG(2000)-FMP2, DSPE-PEG(2000)-VBP, DSPE-PEG(2000)-CBP, DSPE-mPEG(1000), and DSPE-Rhodamine B were dissolved in 1:1 chloroform/methanol and mixed at various mole percentages. The organic solvent was removed under vacuum (Rotovap) to form a thin lipid film, followed by additional drying under a stream of nitrogen. The lipid film was then reconstituted in saline followed by at least 10 freeze-thaw cycles and subsequent extrusion through either a 100 nm or 200 nm pore size filter (10 mL Lipex Extruder) at least 5 times to obtain particles of the desired size.

TABLE 1
Summary of Synthetic Platelet Particles
Containing FMP1 vs. FMP2

| | | | Mole % |
|---|---|---|---|
| | | | FMP of |
| Particle # | FMP# | FMP Sequence | total lipids |
| Particle 1 | FMP1 | cyclo-{Pra}CNPRGD{Tyr(OEt)}RC) | 15% |
| Particle 2 | FMP1 | cyclo-{Pra}CNPRGD{Tyr(OEt)}RC) | 2.5% |
| Particle 3 | FMP2 | Cyclo-(CNPRGD{Tyr(OEt)}R-β-A) | 15% |
| Particle 4 | FMP2 | Cyclo-(CNPRGD{Tyr(OEt)}R-β-A) | 2.5% |

Resultant particles were characterized for their physicochemical properties, namely particle size and surface charge using a Zetasizer (Anton Paar LiteSizer™ 500). Synthetic platelets containing FMP2 were evaluated to ensure that particle size and charge were comparable (within +/−20%) to those containing FMP1 and that this change in surface chemistry did not induce particle aggregation over time (15 weeks). Furthermore, the functionality of these peptide-decorated particles was examined using two methods: platelet aggregometry and flow cytometry. For both assays, human whole blood was drawn from healthy, aspirin-refraining donors via venipuncture into a syringe containing 3.8% w/v sodium citrate. For platelet aggregometry, platelet rich plasma (PRP) was obtained by centrifugation of whole blood in tabletop centrifuge at 150×g for 15 minutes. Half of the PRP was reserved while the remaining was centrifuged again at 13,000×g for 5 minutes to obtain platelet free plasma (PFP). PRP platelet count was determined with a Coulter Counter (Multisizer 3 Coulter Counter, Beckman Coulter) and adjusted to 50,000 platelets per L to generate thrombocytopenic platelet rich plasma (tPRP). Platelet aggregation experiments were performed with 225 μL tPRP stimulated with 25 μL of 5 or 10 μM adenosine 5′-diphosphate (ADP) (or saline as a negative control) on a Bio/Data PAP-8E Platelet Aggregation Profiler. Various synthetic platelet particle formulations were added to the tPRP with resting platelets (without ADP) versus activated platelets (with ADP). The final aggregation percentage was reported and normalized to the final aggregation percentage of ADP activated tPRP from the same donor without synthetic platelet particles (Equation 1). This step was necessary to give a more accurate representation of the improvement in final aggregation values due to variation in the blood amongst donors.

$$Final Aggregation \% = \frac{\begin{matrix}Final Aggregation of ADP Activated tPRP + \\ Sample ( SP )\end{matrix}}{Final Aggregation of ADP Activated tPRP} \times 100 \qquad ( Equation 1 )$$

For flow cytometry, platelet rich plasma (PRP) was obtained by centrifugation of whole blood in tabletop centrifuge at 150×g for 15 minutes. To obtain gel-filtered platelets, an Econo-Column® Chromatography Columns was filled ~50% with Sepharose CL-2B beads and used to obtain gel-filtered platelets. The column was rinsed with HEPES Tyrode's buffer, and then rinsed with Supplemented HEPES Tyrode's buffer (1% BSA, 5 mM D-Glucose). The PRP was diluted with acid citrate dextrose (0.3% v/v). The PRP was added to the column and aliquots were collected. Cloudy solutions were combined and used for the sample preparation. Samples were prepared with 5 μL FITC anti-CD41a and 5 μL AlexaFluor 647 anti-CD62P. Platelets were activated with 5 μM TRAP or 10 μM ADP (or left unactivated) for 20 minutes at 25° C. The samples were then diluted to 1 mL with Tyrode's Solution (Modified I, Boston BioProducts, Inc.) and transferred to FACS tubes. The samples were evaluated on a BD-LSRll. The laser parameter voltages were set as follows (FSC: 625, SSC:375, B525: 520, R660:650, YG582: 694, YG610:490). Two lasers were set for Rhodamine-B and the YG582 was used for analysis. Data analysis was done using FlowJo™ and GraphPad Prism®. Samples were gated for the entire cell population and then for the single cell population. The FITC-CD41a stained cells were gated to determine the platelet population. Within this population the mean of Rhodamine-B labeled particles binding to all platelets (CD41a stains) and activated platelets (AlexaFluor 647 anti-CD62P) were determined. Mean fluorescence and standard deviation (CV) were determined using FlowJo™ software, and data analysis was performed using GraphPad Prism®. Altogether, these assays demonstrated that the functionality (i.e., binding to GPIIb/IIIa on active platelets and enhancement of activated platelet aggregation) of FMP2-decorated synthetic platelets is retained. To account for both inter- and intra-lot and assay variability, three (3) separate manufacturing lots of each synthetic platelet formulation were evaluated for physicochemical and functional characteristics. For functional testing, experiments were performed on at least three different days using blood from different donors. The mean value and standard deviation across runs of each assay were calculated in Microsoft Excel® or GraphPad Prism®. Statistically significant differences were determined using Welch's two-tailed T-Test and a 95% confidence interval in the GraphPad Prism software. Results were considered statistically significant with p<0.05.

### C. Results

### Peptide Functionality Evaluation

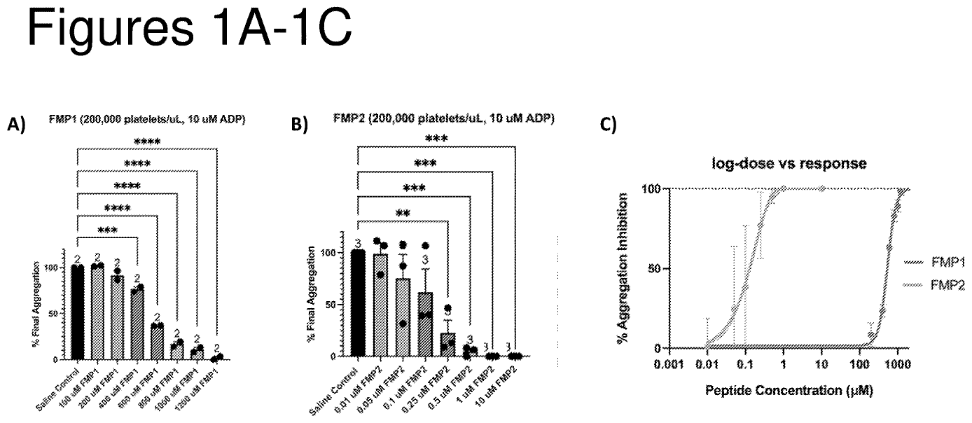

The ability of each peptide to inhibit platelet aggregation was tested over a range of concentrations from 0.0001 μM to 250 μM to determine the IC₅₀ (FIGS. 1A-1B). FMP2 had an approximate IC₅₀ of 0.13 μM. FMP1 peptide had an approximate IC₅₀ of 536.6 μM.

### MALDI-TOF Evaluation of Peptides and Lipid-Peptide Conjugates

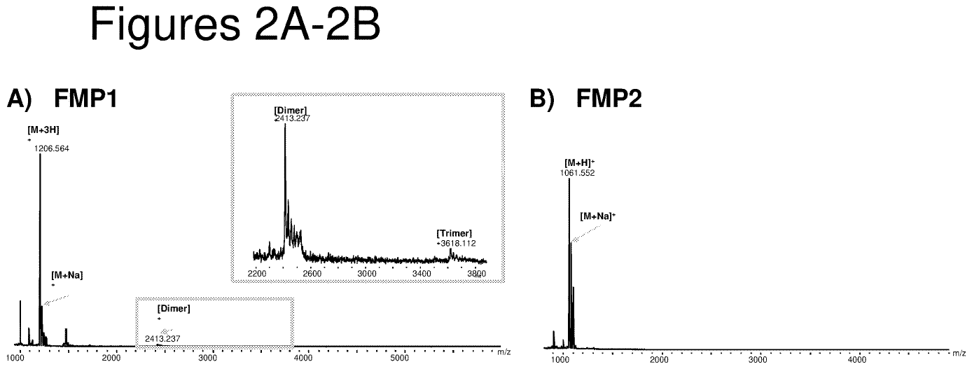

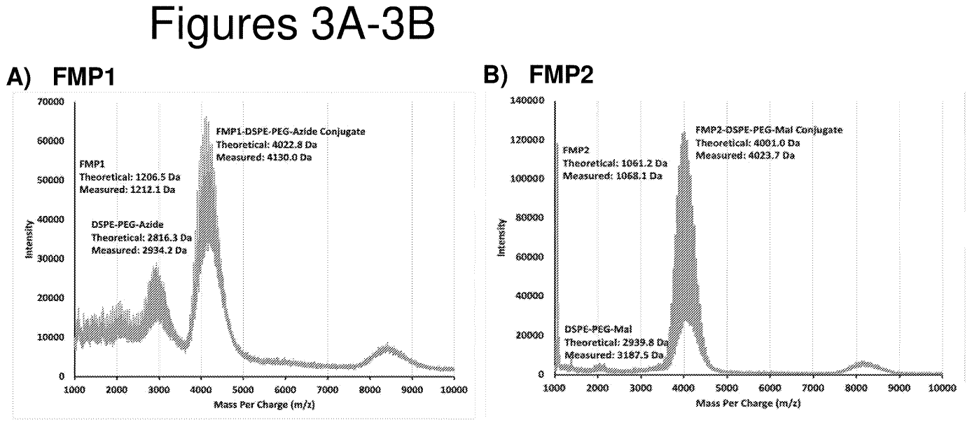

The mass spectrum results for FMP1 vs. FMP2 are shown in FIGS. 2A-2B. FMP1 shows evidence of dimer/trimerization, likely due to cyclic disulfide bond breaking and reforming with free sulfhydryls on neighboring peptides. Dimer/trimerization was not seen in FMP2 samples, suggesting improved stability. Furthermore, the mass spectrum results for the lipid-peptide conjugates are shown in FIGS. 3A-3B. Results confirm that FMP1 and FMP2 are effectively conjugated to DSPE-PEG(2000). A summary of actual measured peaks vs. expected theoretical molecular weights is shown in Table 2, demonstrating that all peptides and lipid-peptide conjugates were within 10% of the expected theoretical values. Overall, MALDI-TOF confirms the conjugation of both FMP1 and FMP2 to their respective lipids.

TABLE 2
Theoretical vs. Measured by MALDI-TOF Molecular Weights
of FMP Peptides and Lipid-Peptide Conjugates

| | | Actual | |
|---|---|---|---|
| | Theoretical | Measured | |
| Peptide/Lipid/ | Molecular | Molecular | % |
| Conjugate | Weight (Da) | Weight (Da) | Difference |
| FMP1 | 1206.5 | 1206.6 | 0.0% |
| DSPE-PEG(2000)-FMP1 | 4022.8 | 4130.0 | 2.7% |
| Conjugate | | | |
| FMP2 | 1061.2 | 1061.5 | 0.0% |
| DSPE-PEG(2000)-FMP2 | 4001.0 | 4023.7 | 0.6% |
| Conjugate | | | |

### Synthetic Platelet Physicochemical Characterization

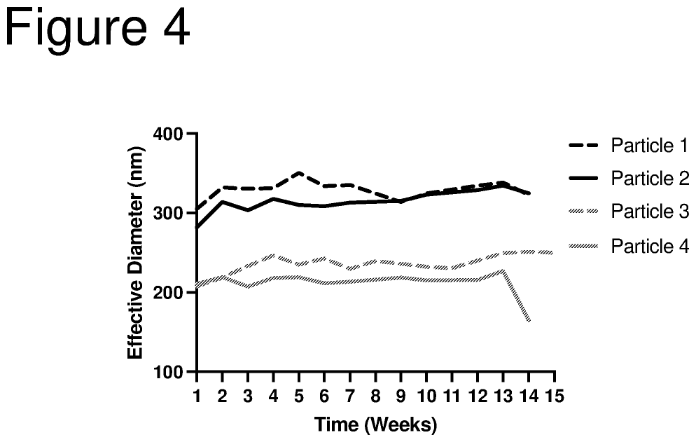

The size and zeta potential characterization of synthetic platelet particle formulations immediately following manufacture (Table 3) and over a 15-week storage period at 25° C. were recorded (FIG. 4). The particles made with FMP2 at both 2.5 mole % (Particle 4) and 15 mole % (Particle 3) retained their size within the desired range (150-250 nm diameter), while particles with FMP1 at both 2.5 mole % (Particle 2) and 15 mole % (Particle 1) showed higher than expected starting size range (>250 nm diameter), perhaps indicating particle aggregation. Welch's t-test for all formulations included in the study showed Particle2 and Particle4 to be not significant (p=0.0542) and formulations Particle1 and Particle3 to be significantly different (p=0.0100). Polydispersity index (PDI) of Particle 4 is greater than 0.3, suggesting particle aggregation.

TABLE 3
DLS Starting Effective Diameter, Number Mean Diameter
and Intensity Mean Diameter and Zeta Potential

| | | Starting | Starting | | |
|---|---|---|---|---|---|
| | Starting | Number | Intensity | | |
| | Effective | Mean | Mean | Starting | Zeta |
| Particle # | Diameter | Diameter | Diameter | PDI | Potential |
| Particle 1 | 281.7 ± 109.1 | 121.8 ± 99.2 | 357.9 ± 491.1 | 0.15 | −7.4 ± 0.8 |
| Particle 2 | 313.9 ± 148.9 | 209.1 ± 130.1 | 471 ± 292.3 | 0.225 | −15.8 ± 0.5 |
| Particle 3 | 234.9 ± 114.9 | 192.7 ± 123.7 | 316.9 ± 155.2 | 0.238 | −12.5 ± 0.6 |
| Particle 4 | 227.3 ± 69.3 | 69.1 ± 26.7 | 242.7 ± 42.7 | 0.93 | −19.4 ± 0.8 |

### Synthetic Platelet Particle Functional Evaluation Via Platelet Aggregometry

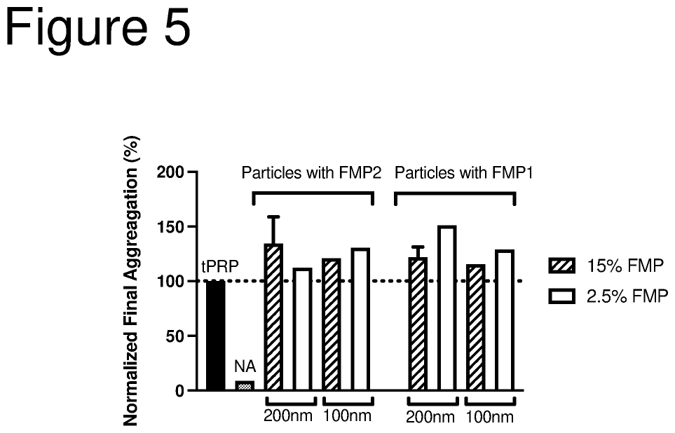

The percent final platelet aggregation (calculated using Equation 1) is summarized in FIG. 5. Synthetic platelet formulations containing FMP2 showed an increase in final aggregation compared to tPRP samples without synthetic platelets, and the level of increase was comparable to particles containing FMP1 (p=0.3986, not significant).

### Synthetic Platelet Particle Functional Evaluation Via Flow Cytometry

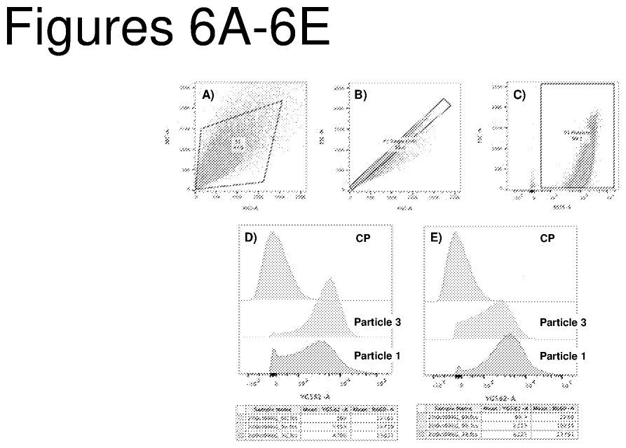

The gating strategies and fluorescence histograms for ADP and TRAP activated samples are shown in FIGS. 6A-6E. The flow cytometry results showed low binding of control particles (CP, lipid particles without peptide decoration) to unactivated, ADP activated and TRAP activated platelets. Synthetic platelet particle formulations with FMP1 (Particle 1) and FMP2 (Particle 3) showed significant binding (p<0.05 compared to CP group) to TRAP and ADP activated platelets.

### D. Summary/Conclusions

A summary of these characterization studies including the experimental methods, test articles, pass fail criteria, and results are tabulated below in Table 4.

TABLE 4
Study Summary

| | Experimental | Test Articles and | | |
|---|---|---|---|---|
| Study End Point | Methods | Controls | Pass Fail Criteria | Results |
| Peptide Inhibition | Platelet Lumi- | FMP1 | Inhibition of | Pass |
| of Platelet | Aggregometry | FMP2 | Platelet | |
| Aggregation | | | Aggregation, | |
| | | | FMP2 IC₅₀ ≤ | |
| | | | FMP 1 IC₅₀ | |
| Peptide | MALDI-TOF | FMP1 | Detection of | Pass |
| Conjugation | | FMP2 | Peptide-Lipid | Pass |
| Confirmation | | | Conjugate | |
| Particle Size | Dynamic Light | Synthetic platelet | Particle effective | |
| | Scattering (DLS) | particles with | diameter | |
| | | FMP1: Particle 1 | maintained over | Pass |
| | | Particle 2 | 15 weeks storage | Pass |
| | | Synthetic platelet | at room | |
| | | particles with | temperature | |
| | | FMP2: | | |
| | | Particle 3 | | Pass |
| | | Particle 4 | | Pass |
| Particle charge | Zeta Potential | Synthetic platelet | Zeta | |
| | | particles with | potential < −5 mV | |
| | | FMP1: Particle 1 | | Pass |
| | | Particle 2 | | Pass |
| | | Synthetic platelet | | |
| | | particles with | | |
| | | FMP2: | | |
| | | Particle 3 | | Pass |
| | | Particle 4 | | Pass |
| Platelet | Platelet Lumi- | Synthetic platelet | Final Aggregation | |
| Aggregation | Aggregometry | particles with | of tPRP + | |
| Functionality | | FMP1: Particle 1 | particles > tPRP | Pass |
| | | Particle 2 | alone | Pass |
| | | Synthetic platelet | | |
| | | particles with | | |
| | | FMP2: | | |
| | | Particle 3 | | Pass |
| | | Particle 4 | | Pass |
| Activated | Flow Cytometry | Synthetic platelet | Synthetic platelet | |
| Platelet | | particles with | binding > Control | |
| Binding | | FMP1: Particle 1 | particle binding | Pass |
| | | Synthetic platelet | | |
| | | particles with | | |
| | | FMP2: Particle 3 | | Pass |

Overall, the data suggest that FMP2 inhibits aggregation of platelets at concentrations over three orders of magnitude below FMP1, indicating that the potency of the peptide is significantly improved by the changes in chemistry. FMP2 also demonstrates greater stability than FMP 1 as indicated by the lack of formation of dimers/trimers. Synthetic platelet particles incorporating FMP2 demonstrate similar and in some cases improved physicochemical and functional properties compared to particles containing FMP1.

### REFERENCES

[1] Cheng, S. et al., Craig, “Design and Synthesis of Novel Cyclic RGD-Containing Peptides as Highly Potent and Selective Integrin αIIbβ3 Antagonists. J. Med. Chem., 37(1): 1-8 (1994).
[2] Yee, D. et al., “Aggregometry detects platelet hyperreactivity in healthy individuals,” Blood, 106: 2723-2729 (2005).

### Example 2: Various Synthetic Platelet Formulations

### A. Study Purpose and Background

Various synthetic platelet formulation compositions were evaluated based on size, surface charge, stability, and functionality. Briefly, molar ratios of components were varied as shown in Table 5. Physicochemical characterization of synthetic platelet formulations consisted of dynamic light scattering and zeta potential measurements using an Anton Parr Litesizer 500, size and morphology assessed by Cryo-TEM, and concentration and yield determined by a Biotek Synergy M5 fluorescent plate reader. Stability of the selected formulations from the physiochemical analysis was assessed by size and charge using Anton Parr Litesizer 500 on samples in simulated storage and transportation conditions including room temperature, 4° C., cycling between 4° C. and room temperature, and cold packs with and without agitation. Functionality characterization of the selected formulations from the physicochemical analysis was assessed by binding to activated platelets in flow cytometry using a BD Accuri B6 cytometer, platelet aggregometry using a BioData PAP-8E LTA, collagen/vWF binding and platelet recruitment under flow using BioFlux, clot formation time in ROTEM-NATEM, and in vivo bleeding time/blood loss assessment in a tail transection model in thrombocytopenic mice.

TABLE 5
Synthetic Platelet Formulation Compositions in Molar % of Total Lipid Content

| | | | | | DSPE- | DSPE- | | | | |
|---|---|---|---|---|---|---|---|---|---|---|
| | DSPE-PEG(2000)-PBP | | | | PEG | PEG | DSPE- | | | |
| | Molar % | | | | (2000)- | (2000)- | mPEG | | | DSPE- |
| | | | | P- | CBP | VBP | (1000) | DSPC | Cholesterol | Cy5 |
| | FM | IRG | Fg- | select | Molar | Molar | Molar | Molar | Molar | Molar |
| Particle # | P2 | D | H12 | in | % | % | % | % | % | % |
| Particle | 2.50 | — | — | — | 1.25 | 1.25 | 2.5 | 46.5 | 45.0 | 1.0 |
| 1 | | | | | | | | | | |
| Particle | 0.50 | — | — | — | 1.25 | 1.25 | 4.5 | 46.5 | 45.0 | 1.0 |
| 2 | | | | | | | | | | |
| Particle | 2.50 | — | — | — | 1.25 | 0.25 | 3.5 | 46.5 | 45.0 | 1.0 |
| 3 | | | | | | | | | | |
| Particle | 2.50 | — | — | — | 0.25 | 1.25 | 3.5 | 46.5 | 45.0 | 1.0 |
| 4 | | | | | | | | | | |
| Particle | 0.50 | — | — | — | 0.25 | 0.25 | 6.5 | 46.5 | 45.0 | 1.0 |
| 5 | | | | | | | | | | |
| Particle | 1.25 | — | — | — | 2.50 | 1.25 | 2.5 | 46.5 | 45.0 | 1.0 |
| 6 | | | | | | | | | | |
| Particle | 1.25 | — | — | — | 1.25 | 2.50 | 2.5 | 46.5 | 45.0 | 1.0 |
| 7 | | | | | | | | | | |
| Particle | 2.50 | — | — | — | 0.25 | 0.25 | 4.5 | 46.5 | 45.0 | 1.0 |
| 8 | | | | | | | | | | |
| Particle | 2.50 | — | — | — | 1.25 | — | 3.8 | 46.5 | 45.0 | 1.0 |
| 9 | | | | | | | | | | |
| Particle | 2.50 | — | — | — | — | 1.25 | 3.8 | 46.5 | 45.0 | 1.0 |
| 10 | | | | | | | | | | |
| Particle | 1.50 | — | — | — | 0.75 | 0.75 | 4.5 | 46.5 | 45.0 | 1.0 |
| 11 | | | | | | | | | | |
| Particle | 1.00 | — | — | — | 0.50 | 0.50 | 5.5 | 46.5 | 45.0 | 1.0 |
| 12 | | | | | | | | | | |
| Particle | 1.5 | — | — | — | — | — | 6.0 | 46.5 | 45.0 | 1.0 |
| 14 | | | | | | | | | | |
| Particle | 1.0 | — | — | — | — | — | 6.5 | 46.5 | 45.0 | 1.0 |
| 15 | | | | | | | | | | |
| Particle | 0.5 | — | — | — | — | — | 7.0 | 46.5 | 45.0 | 1.0 |
| 16 | | | | | | | | | | |
| Particle | — | — | — | — | 0.5 | — | 7.5 | 46.5 | 45.0 | 1.0 |
| 18 | | | | | | | | | | |
| Particle | — | — | — | — | — | 0.5 | 7.5 | 46.5 | 45.0 | 1.0 |
| 19 | | | | | | | | | | |
| Particle | 0.10 | — | — | — | 0.05 | 0.05 | 7.3 | 46.5 | 45.0 | 1.0 |
| 20 | | | | | | | | | | |
| Particle | 0.10 | — | — | — | 0.5 | 0.5 | 6.4 | 46.5 | 45.0 | 1.0 |
| 21 | | | | | | | | | | |
| Particle | 1.0 | — | — | — | — | 0.5 | 6.0 | 46.5 | 45.0 | 1.0 |
| 22 | | | | | | | | | | |
| Particle | 1.0 | — | — | — | 0.5 | — | 6.0 | 46.5 | 45.0 | 1.0 |
| 23 | | | | | | | | | | |
| Particle | — | — | — | — | 0.5 | 0.5 | 6.5 | 46.5 | 45.0 | 1.0 |
| 24 | | | | | | | | | | |
| Particle | — | 1.0 | — | — | 0.5 | 0.5 | 5.5 | 46.5 | 45.0 | 1.0 |
| 26 | | | | | | | | | | |
| Particle | — | — | 1.0 | — | 0.5 | 0.5 | 5.5 | 46.5 | 45.0 | 1.0 |
| 27 | | | | | | | | | | |
| Particle | — | — | — | 1.0 | 0.5 | 0.5 | 5.5 | 46.5 | 45.0 | 1.0 |
| 28 | | | | | | | | | | |

FMP2 = cyclo-(CNPRGD{Tyr(OEt)} R-β-A)
P-selectin = DAEWVDVS
IRGD = GRGDSP
CBP = [GPO]₇
Fg-H12 = HHLGGAKQAGDV VBP = TRYLRIHPQSWVHQI

### B. Methods

### Manufacturing

Synthetic platelet particles were made using standard lipid film rehydration and extrusion methods. Briefly, DSPC, cholesterol, DSPE-PEG(2000)-PBP (FMP2, 1RGD, Fg-H12, or P-selectin), DSPE-PEG(2000)-VBP, DSPE-PEG(2000)-CBP, DSPE-mPEG(1000), and DSPE-Cy5 were dissolved in 1:1 chloroform/methanol and mixed at various mole percentages. The organic solvent was removed under vacuum (Rotovap) to form a thin lipid film, followed by additional drying under a stream of nitrogen. The lipid film was then reconstituted in saline followed by at least 10 freeze-thaw cycles and subsequent extrusion through either a 100 nm or 200 nm pore size filter (10 mL Lipex Extruder) at least 5 times to obtain particles of the desired size.

Duplicate batches with each particle formulation optimization group was produced in order to obtain a mean inter-lot mean and standard deviation. In addition, all samples were analyzed in triplicate in order to obtain an intra-lot mean value and standard deviation.

### Physicochemical Analysis

Particles were diluted in cell culture grade water to a 1 mg/mL solution for size and charge analysis. The effective hydrodynamic diameters, polydispersity indices, mean diameters normalized by intensity and number, and zeta potential were obtained and evaluated using an Anton Parr Litesizer 500. For morphology analysis using cryo-TEM, samples were prepared at 0.5 mg/mL in cell culture grade water, adsorbed onto glow discharged coated copper grids for 2 minutes, blotted with filter paper, and plunge frozen in liquid ethane below the devitrification temperature of −137° C. Samples were imaged between 13,000×-60,000× magnification, and diameters of the particles were measured using the online software ImageJ.

### In Vitro Functionality by Flow Cytometry

Active platelet binding functionality of synthetic platelet formulations was assessed by flow cytometry. Human whole blood was obtained via venipuncture from healthy donors and collected in vacutainers containing 3.8% sodium citrate. To obtain platelet-rich plasma (PRP), whole blood was centrifuged at 120×g for 15 minutes at 25° C. (without brake). The PRP was diluted with by 2-fold with Tyrode's buffer (137 mmol/L NaCl, 12 mmol/L NaHCO₃, 2.0 mmol/L KCl, 0.3 mmol/L Na₂HPO₄, 1 mmol/L MgCl₂, 5 mmol/L HEPES, 5 mmol/L glucose, pH 7.3) and supplemented with 0.03 units/mL apyrase and centrifuged at 100×g for 15 minutes at 25° C. to pellet any contaminating red and white blood cells. PRP was supplemented with 1 μg/mL prostacyclin for 5 minutes and centrifuged at 600×g for 15 minutes at 25° C. for platelet washing. The platelet pellet was gently resuspended in Tyrode's buffer and allowed to equilibrate on the benchtop for 20-30 minutes prior to flow cytometry staining. The washed platelets were aliquoted into triplicate flow cytometry tubes, incubated at 25° C. with Alexa Fluor 647 anti-CD62P, 5 μM of TRAP agonist, and each of synthetic platelet formulations (platelet:particle ratio ~1:1000) for 20 minutes.

Next, the FITC anti-GP1b(alpha) were added to the platelets and incubated for 1 minute prior to reading on the flow cytometer. Platelets were analyzed on a BD Accuri B6 cytometer until 50,000 counts were measured per sample with gating for platelet populations using side scatter (SSC) on the y-axis and forward scatter (FCS) on the x-axis all in log plots. Gating for fluorescent signals was performed with either FITC or Cy5 on the y-axis and FSC on the x-axis all in log plots. Flow cytometry data was analyzed in FlowJo software in order to obtain fluorescent counts of Cy5 labeled synthetic platelet and FITC labeled platelets.

### In Vitro Functionality by Aggregometry

Active platelet aggregation functionality of synthetic platelet formulations was assessed by light transmission aggregometry. Human whole blood was obtained via venipuncture from healthy donors and collected in vacutainers containing 3.8% sodium citrate. To obtain platelet-rich plasma (PRP), whole blood was centrifuged at 120×g for 15 minutes at 25° C. (without brake). Platelet aggregation was performed with 225 μl of 25% diluted PRP stimulated with 25 μl of 10 μM adenosine 5′-diphosphate (ADP) (or saline as a negative control) on a Bio/Data PAP-8E Platelet Aggregation Profiler. 20 μl of each synthetic platelet sample was added to the 25% diluted PRP, and final aggregation percentage was recorded. The final aggregation percentage was reported and normalized to the final aggregation percentage of TRAP/ADP activated (tPRP) from the same donor without particles (Equation 1).

$$Final Aggregation \% = \frac{Aggregation of Sample}{\begin{matrix}Aggregation of ADP Activated \\ Platelet Rich Plasma ( tPRP )\end{matrix}} \times 100 \qquad ( Equation 1 )$$

### In Vitro Functionality by BioFlux

The collagen/vWF binding and platelet recruiting functionality of synthetic platelet formulations was assessed by BioFlux. BioFlux plates (0-200 dyn/cm²) were coated outlet to inlet with 100 μg/mL Equine Chronolog Collagen by adding 50 μL of collagen in outlet well and starting the flow at 30 dyn/cm² for 30 seconds. Next, the flow was stopped and the plate incubated at 37° C. for 1 hour. Then, 600 μL 0.1% (w/v) BSA was added followed by saline to the inlet well and flowed at 30 dyn/cm² for 5-10 mins. Next, wells were washed with 0.1% (w/v) BSA solution for 5-10 mins. Whole blood was obtained via venipuncture from healthy human donors and collected in vacutainers containing 3.8% sodium citrate. To obtain platelet-rich plasma (PRP), whole blood was centrifuged at 120×g for 15 minutes at 25° C. (without brake). To label platelets with Calcein-AM, 2 μL Calcein-AM, with a stock concentration of 1 mg/mL, was added to every 1 mL of PRP solution and incubated for 20 minute at room temperature. Platelets and Cy5-labeled synthetic platelet particles were added at a 1:100 platelet:particle ratio. The platelet and particle binding was recorded as the % of area covered and calculated over the first minute to obtain binding rate.

### In Vitro Functionality by ROTEM-NATEM

Accelerated clotting functionality of synthetic platelet formulations was assessed via ROTEM using the NATEM methodology. Human whole blood was obtained via venipuncture from healthy donors and collected in vacutainers containing 3.8% sodium citrate. Whole blood was centrifuged at 150×g for 15 minutes to obtain platelet rich plasma (PRP) and RBC fractions. Half of the PRP was further centrifuged at 13,000×g for 5 minutes to obtain platelet free plasma (PFP). PRP, PFP and RBCs were recombined at ratios to obtain simulated thrombocytopenic whole blood (tWB) with 10% of normal platelet counts. 20 μL of STARTEM reagent and 2 μL of synthetic platelet particles were added to ROTEM cups, followed by 300 μL tWB to achieve a final ratio of 1:10 platelets:SP. The end points collected were the clot formation time (CFT), clot formation kinetics A10, and alpha angle. The treatment group values were all normalized to the tWB condition without synthetic platelets to minimize the effect of donor-to-donor variability in platelet/clotting function.

### In Vivo Functionality in Thrombocytopenic Mice

Hemostatic functionality of synthetic platelet formulations was assessed after tail transection in thrombocytopenic (TCP) mice. Platelet counts from wild-type C57/BL6J mice were achieved via a retro orbital (RO) blood draw of 0.1 mL for platelet counting via the HemaVet 950. Dosage calculations were performed, and mice were injected intraperitoneally with anti-CD42b (anti-GPIbα) antibody at 0.2 μg/g. 18 hours after antibody injection, platelet counts were again obtained to monitor for induction of thrombocytopenia (~75% average reduction in platelet counts). Synthetic platelet formulations were injected retro-orbitally at 0.1, 1.0, and 10 mg/kg and 5 mL/kg. Fifteen (15) minutes after treatment administration, the tails of the mice were transected 1 mm from the tip with a sharp surgical blade, and immersed in 1200 μL of warm (37° C.) saline. The time taken for bleeding to stop (bleeding time) was recorded. Also, total blood loss was measuring in the saline using a standard hemoglobin assay.

### C. Results

### Size and Charge by Litesizer

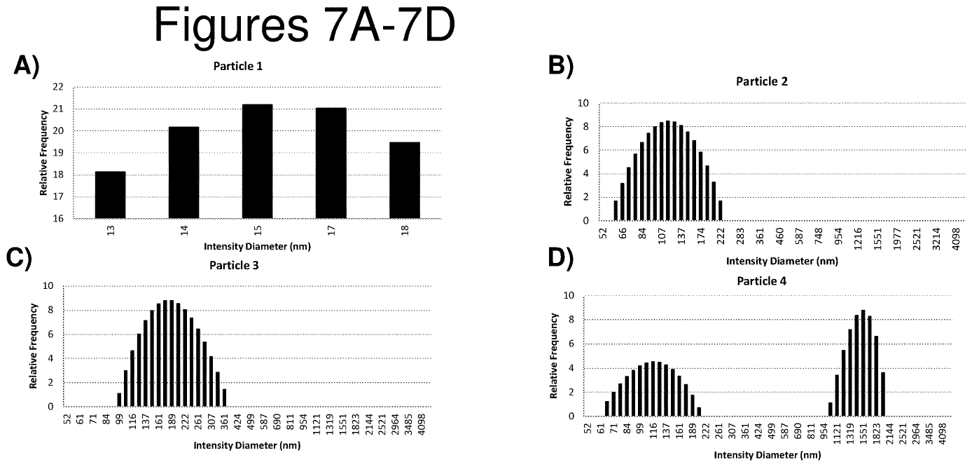

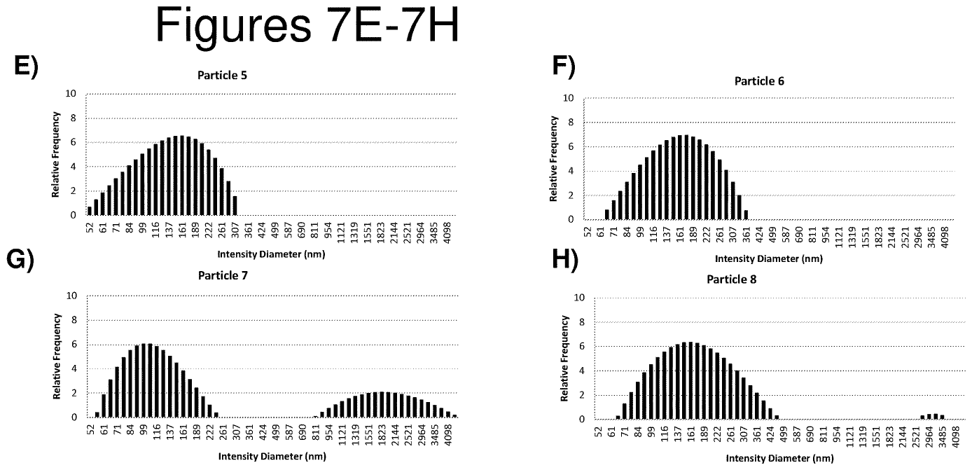

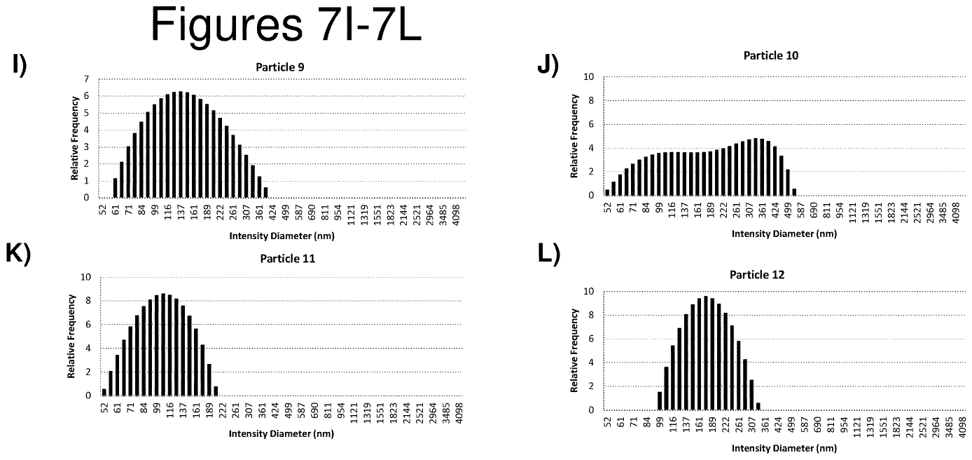

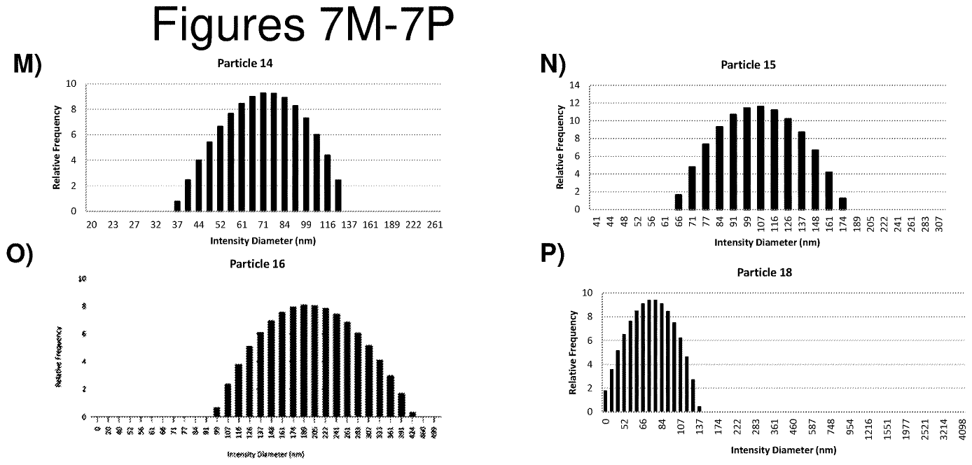

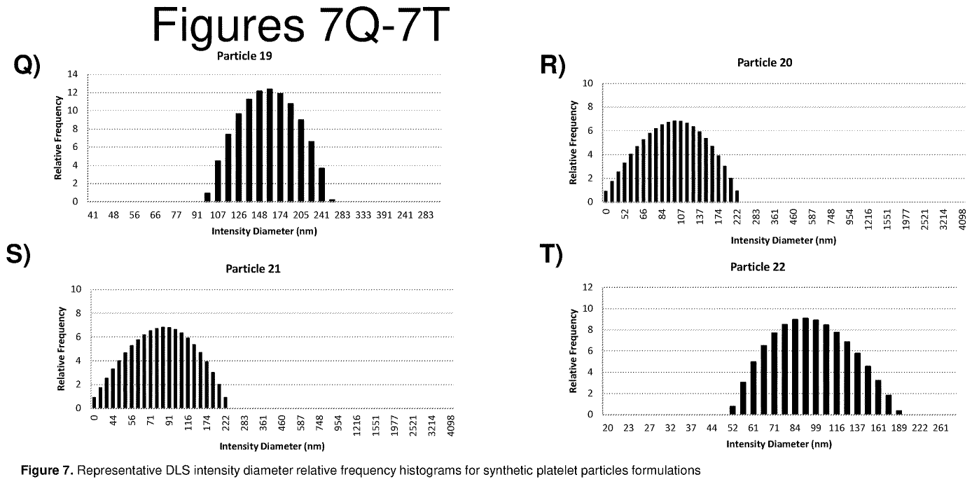

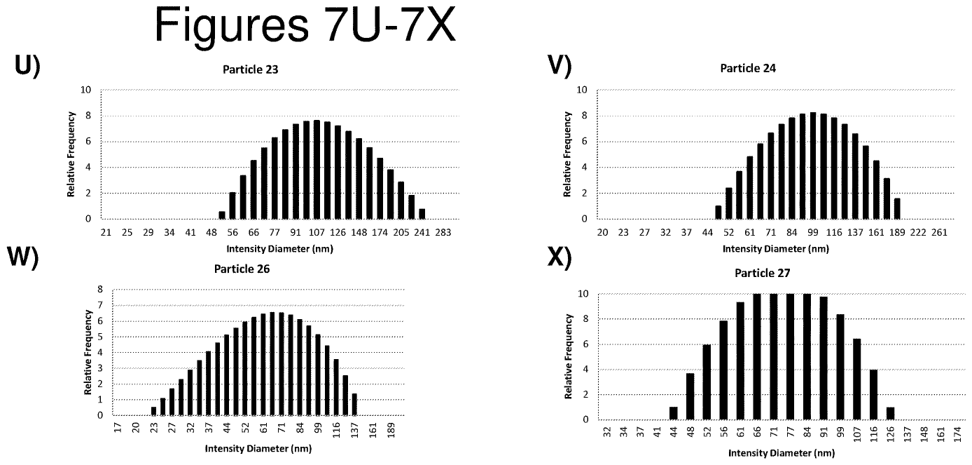

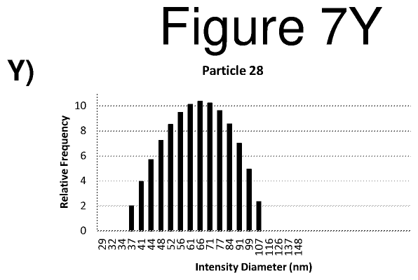

The hydrodynamic diameters and zeta potential of the synthetic platelet formulations were assessed using a Litesizer 500 particle analyzer. The Litesizer 500 used back scatter on a 0.1 mg/mL sample to obtain the hydrodynamic, intensity diameter, polydispersity index (PDI), and zeta potential for all the formulations (Table 6 and FIGS. 7A-7Y). Several particle formulations with high amounts of peptide, >3 mole % of the total lipid content, demonstrated physicochemical instability. Furthermore, several particle formulations with FMP2 only or FMP2 without CBP also demonstrated physicochemical instability.

TABLE 6
DLS and zeta potential measurements by Litesizer
for various synthetic platelet formulations.

| | | | D90 | | |
|---|---|---|---|---|---|
| | Hydrodynamic | Intensity | Intensity | | Zeta |
| | Diameter | Diameter | Diameter | | Potential |
| Particle # | (nm) | (nm) | (nm) | PDI | (mV) |
| Particle 1 | >10,000* | 66 | — | 0.59* | −19 |
| Particle 2 | 132 | 128 | — | 0.22 | −24 |
| Particle 3 | 147 | 148 | — | 0.20 | −35 |
| Particle 4 | >10,000* | 91 | — | 0.54* | −24 |
| Particle 5 | 156 | 162 | 198 | 0.24 | −28 |
| Particle 6 | 143 | 149 | — | 0.23 | −32 |
| Particle 7 | 348* | 130 | — | 0.25 | −21 |
| Particle 8 | 172 | 140 | — | 0.26 | −39 |
| Particle 9 | 138 | 131 | — | 0.13 | −34 |
| Particle 10 | >10,000* | 155 | — | 0.42* | −15 |
| Particle 11 | 159 | — | — | 0.23 | −16 |
| Particle 12 | 151 | 147 | 217 | 0.22 | −21 |
| Particle 14 | 85 | 75 | 103 | 0.18 | −17 |
| Particle 15 | 187 | 148 | 214 | 0.3* | −7 |
| Particle 16 | 187 | 148 | — | 0.36* | −7 |
| Particle 18 | 217 | 202 | 275 | 0.19 | −49 |
| Particle 19 | 171 | 145 | 214 | 0.23 | −1* |
| Particle 20 | 193 | 145 | 221 | 0.25 | −14 |
| Particle 21 | 101 | 74 | 98 | 0.3* | −6 |
| Particle 22 | 152 | 113 | 177 | 0.32* | −19 |
| Particle 23 | 195 | 171 | 247 | 0.27 | −17 |
| Particle 24 | 147 | 102 | 146 | 0.29 | −5 |
| Particle 26 | 80 | 66 | 102 | 0.27 | −12 |
| Particle 27 | 95 | 77 | 100 | 0.17 | −0.3* |
| Particle 28 | 89 | 66 | 88 | 0.21 | −35 |

*indicates values outside of desired range.
“—” indicates data not available.

### Morphology and Size by Cryo-TEM

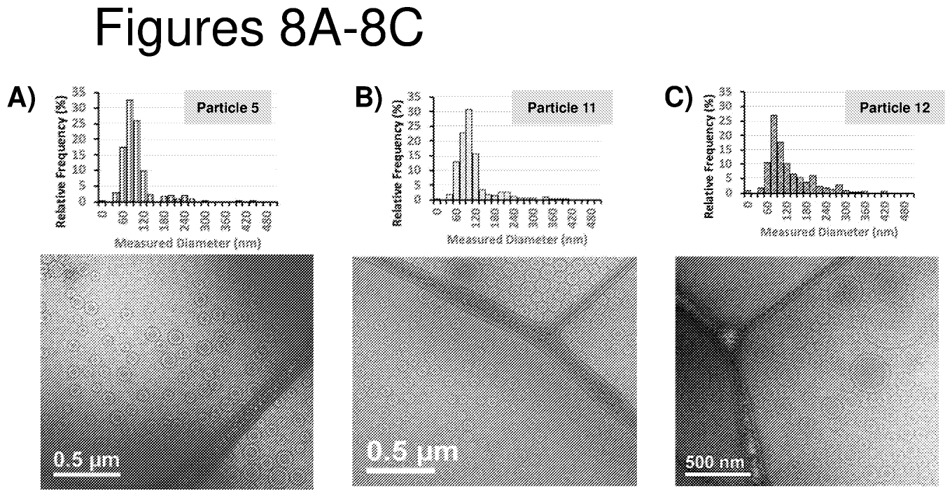

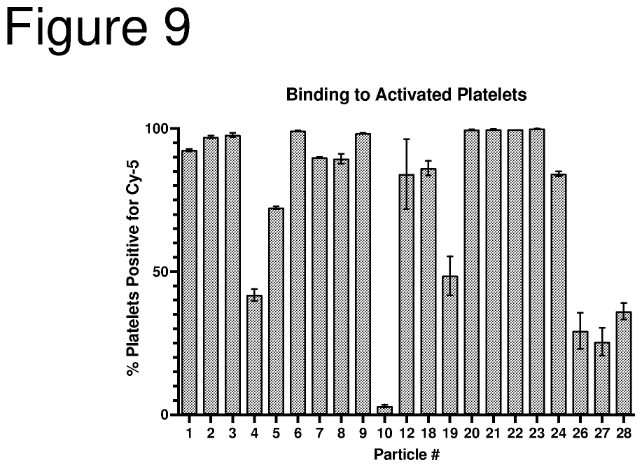

The morphology and size of several of the synthetic platelet formulations were evaluated by Cryo-TEM. As shown in Table 7 and FIGS. 8A-8C, all three formulations that were evaluated had an unilamellarity percentage above 8500. The size distributions and PDI values of the measured particles by Cryo-TEM were comparable to the Litesizer measurements for hydrodynamic diameter and intensity diameters.

TABLE 7
Cryo-TEM particle size and unilamellarity measurements
by Image J for various synthetic platelet formulations.

| | Mean | Maximum | | |
|---|---|---|---|---|
| | Diameter | Diameter | | Unilamellarity |
| Particle # | (nm) | (nm) | PDI | (%) |
| Particle 5 | 99 | 499 | 0.27 | 85 |
| Particle 11 | 110 | 399 | 0.26 | 87 |
| Particle 12 | 121 | 412 | 0.27 | 87 |

### In Vitro Functionality by Flow Cytometry

The percentage of activated platelet population that was positive for Cy5-labeled particle binding is summarized in FIG. 9 for various synthetic platelet formulations. Overall, formulations that contain platelet binding peptide FMP2 showed higher binding to activated platelets compared to formulations containing 1RGD, Fg-H12 or P-selectin binding peptides.

### In Vitro Functionality by Aggregometry

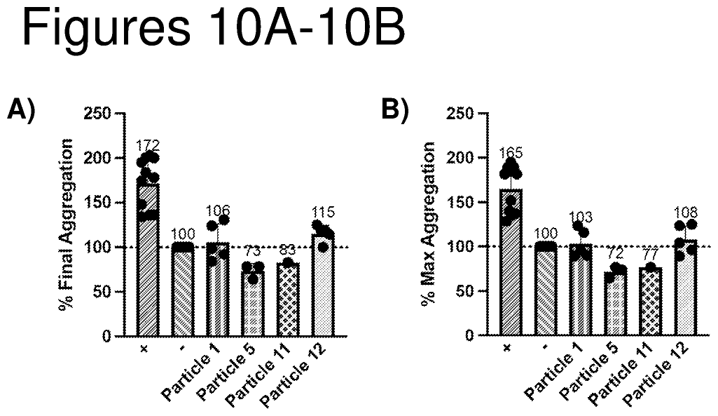

The percentage of final aggregation and percentage of max aggregation of PRP samples with platelet deficiency (i.e., thrombocytopenia, TCP) containing various synthetic platelet formulations relative to samples without synthetic platelets was calculated and recorded, as shown in FIGS. 10A-10B. In this aggregation assay, only Particle 12 demonstrated an increase in both the percentage of final aggregation (115%) and max aggregation (108%) in comparison to the platelet defect. For all formulations, the increase in aggregation was not significantly different from the platelet deficiency defect.

### In Vitro Functionality by ROTEM-NATEM

A summary of the ROTEM-NATEM analysis of various synthetic platelet formulations in human whole blood (WB) with 10% platelets (i.e., platelet deficiency) is summarized in Table 8. Particle 12 and 20 demonstrated significant (p<0.05) rescue in clotting kinetics parameters including clot formation time (CFT), amplitude of clot curve at 10 minutes (A10), and alpha angle compared to samples that were treated with buffer. Particle 23 demonstrated significant delay in clotting kinetics compared to buffer group (p<0.05).

TABLE 8
Summary of ROTEM-NATEM analysis of whole blood (WB) with 10% platelets
supplemented with various synthetic platelet formulations

| | | WB | | | | | | | | | | |
|---|---|---|---|---|---|---|---|---|---|---|---|---|
| | | with | WB with 10% Platelets | | | | | | | | | |
| | | 100% | | Particle | Particle | Particle | Particle | Particle | Particle | Particle | Particle | Particle |
| | | Platelets | Buffer | 5 | 12 | 18 | 19 | 20 | 21 | 22 | 23 | 24 |
| CFT | Mean | 63.45 | 100 | 107.2 | 85.27* | 81.67 | 91.68 | 77.54* | 89.2 | 99.15 | 119.9+ | 109.5 |
| | SEM | 2.003 | 0 | 7.456 | 4.254 | 7.687 | 3.764 | 6.067 | 7.011 | 9.941 | 7.012 | 19.68 |
| A10 | Mean | 128.9 | 100 | 99.82 | 111.2* | 106.1 | 102.1 | 102 | 98.22 | 99.06 | 91.4 | 89.83 |
| | SEM | 2.249 | 0 | 3.031 | 4.609 | 3.521 | 2.083 | 2.25 | 1.908 | 1.68 | 6.228 | 11.75 |
| alpha | Mean | 119.66 | 100 | 97.32 | 111.9* | 109.8 | 106.1 | 111.7* | 105.1 | 101.1 | 89.48 | 95.16 |
| | SEM | 1.943 | 0 | 3.674 | 3.545 | 3.265 | 3.44 | 3.422 | 3.834 | 5.825 | 3.111 | 8.833 |

*significant rescue compared to buffer group (p < 0.05)
+significant delay compared to buffer group (p < 0.05)

### In Vitro Functionality by BioFlux

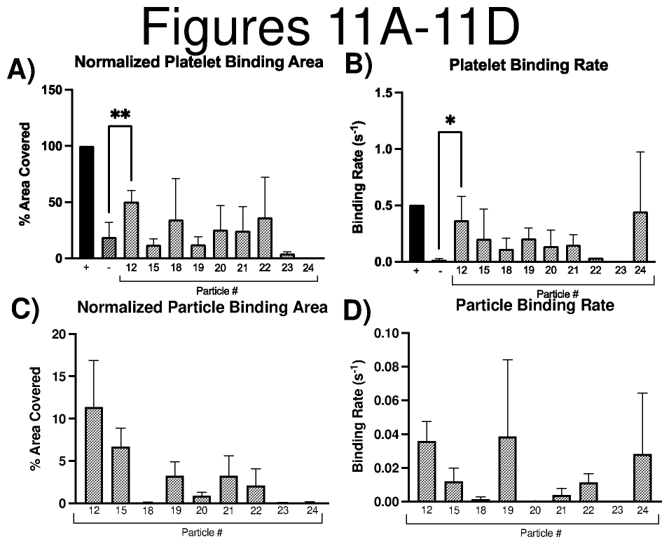

Synthetic platelet particle binding to collagen/vWF surfaces and platelet recruitment onto the surface was assayed using BioFlux. The platelet and particle binding area (% coverage) and rate for various synthetic platelet formulations is summarized in FIGS. 11A-11D. In this assay, only Particle 12 demonstrated significant increase in platelet recruitment, both area covered and binding rate, onto the collagen/vWF surface compared to the negative control (vehicle, *p<0.05 and **p<0.01). Particle 12 also demonstrated substantial adhesion to the vWF/collagen surface.

### In Vivo Functionality in Thrombocytopenic Mice

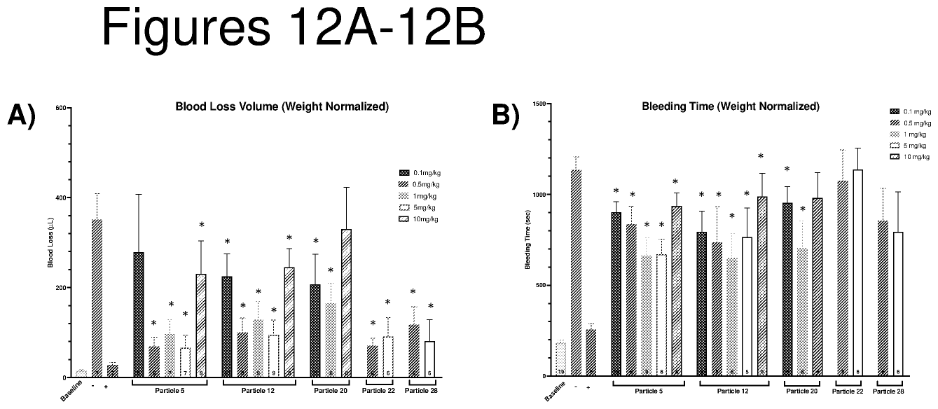

Several synthetic platelet formulations were evaluated for hemostatic efficacy after tail transection in thrombocytopenic mice. Blood loss volume and bleeding time were recorded, as summarized in FIGS. 12A-12D. All formulations tested demonstrated significant reduction in total blood loss at doses between 0.1-10 mg/kg (*p<0.05). Bleeding time was also reduced in animals treated with Particle 5, Particle 12, and Particle 20. Overall, maximum efficacy was seen in doses between 0.5-5 mg/kg.

### D. Summary/Conclusions

A summary of these physicochemical and functional characterization study results are tabulated below in Table 9.

TABLE 9
Summary of synthetic platelet physicochemical and functional evaluation.

| | | | | | | | | Mouse |
|---|---|---|---|---|---|---|---|---|
| | Hydro- | | | Flow | | | | TCP |
| | dynamic | | Zeta | Cytometry | | ROTEM- | BioFlux | Efficacy |
| | Diameter | | Potential | Platelet | Platelet | NATEM | Platelet | Window |
| Formulation | (nm) | PDI | (mV) | Binding | Aggregation | (CFT, alpha) | Recruitment | (mg/kg) |
| Particle 1 | >10,000 | 0.59 | −19 | High | Increased | — | — | — |
| | | | | | aggregation | | | |
| Particle 2 | 132 | 0.22 | −24 | High | — | — | — | — |
| Particle 3 | 147 | 0.20 | −35 | High | — | — | — | |
| Particle 4 | >10,000 | 0.54 | −24 | Medium | — | — | — | — |
| Particle 5 | 156 | 0.24 | −28 | High | Decreased | No significant | — | 0.5-5.0 |
| | | | | | aggregation | effect | | mg/kg |
| Particle 6 | 143 | 0.23 | −32 | High | — | — | — | — |
| Particle 7 | 348 | 0.25 | −21 | High | — | — | — | — |
| Particle 8 | 172 | 0.26 | −39 | High | — | — | — | — |
| Particle 9 | 138 | 0.13 | −34 | High | — | — | — | — |
| Particle 10 | >10,000 | 0.42 | −15 | Low | — | — | — | — |
| Particle 11 | 159 | 0.23 | −16 | High | Decreased | — | — | — |
| | | | | | aggregation | | | |
| Particle 12 | 151 | 0.22 | −21 | High | Increased | Significant | Significant | 0.5-5.0 |
| | | | | | aggregation | rescue in | increase | mg/kg |
| | | | | | | CFT, A10 and | in platelet | |
| | | | | | | alpha | binding | |
| Particle 14 | 85 | 0.18 | −17 | — | — | — | — | — |
| Particle 15 | 187 | 0.3 | −7 | — | — | — | No | — |
| | | | | | | | significant | |
| | | | | | | | effect | |
| Particle 16 | 187 | 0.36 | −7 | — | — | — | — | — |
| Particle 18 | 217 | 0.19 | −49 | High | — | No significant | No significant | — |
| | | | | | | effect | effect | |
| Particle 19 | 171 | 0.23 | −1 | Medium | — | No significant | No | — |
| | | | | | | effect | significant | |
| | | | | | | | effect | |
| Particle 20 | 193 | 0.25 | −14 | High | — | Significant | significant | 0.5-1 |
| | | | | | | rescue in CFT | No effect | mg/kg |
| | | | | | | and alpha | | |
| Particle 21 | 101 | 0.3 | -6 | High | — | No significant | No | — |
| | | | | | | effect | significant | |
| | | | | | | | effect | |
| Particle 22 | 152 | 0.32 | −19 | High | — | No significant | No | 0.5-5 |
| | | | | | | effect | significant | mg/kg (blood |
| | | | | | | | effect | loss only) |
| Particle 23 | 195 | 0.27 | −17 | High | — | Significant | No | — |
| | | | | | | delay in CFT | significant | |
| | | | | | | | effect | |
| Particle 24 | 147 | 0.29 | −5 | High | — | No significant | No | — |
| | | | | | | effect | significant | |
| | | | | | | | effect | |
| Particle 26 | 80 | 0.27 | −12 | Medium | — | — | — | — |
| Particle 27 | 95 | 0.17 | −0.3 | Medium | — | — | — | — |
| Particle 28 | 89 | 0.21 | −35 | Medium | — | — | — | 0.5-5 |
| | | | | | | | | mg/kg |
| | | | | | | | | (blood loss |
| | | | | | | | | only) |

″—″ indicates data not available.

Overall, the selection of platelet binding peptide and ratios between various peptides in the formulation impact the physicochemical and functional properties of synthetic platelet formulations.

### REFERENCES

[1] Danei, M. Dehghankhold, S. Ataei, F. Hasanzadeh Davarani, R. Javanmard, A. Dokhani, S. Khorasani and M. R. Mozafari. Impact of Particle Size and Polydispersity Index on the Clinical Applications of Lipidic Nanocarrier Systems. Pharmaceutics (2018) Vol 10, Issue 57, 1-17.
[2] FDA Guidance for Liposomal Drug Products. U.S. Department of Health and Human Services Food and Drug Administration Center for Drug Evaluation and Research (CDER). April 2018.
[3] Wang, Y. Grainger, D. W. Lyophilized liposome-based parenteral drug development: Reviewing complex product design strategies and current regulatory environments. Advanced Drug Delivery Reviews (2019) Vol 151-152. pg 56-71.
[4] Tang, X. M. J. Pikal, Design of freeze-drying processes for pharmaceuticals: practical advice, Pharm. Res. 21 (2004) 191-200.
[5] Sylvester, B. A. Porfire, M. Achim, L. Rus, I. Tomuti, A step forward towards the development of stable freeze-dried liposomes: a quality by design approach (QbD), Drug Dev. Ind. Pharm. 44 (2018) 385-397.
[6] Iyer, Lavanya K et al. “Process and Formulation Effects on Protein Structure in Lyophilized Solids Using Mass Spectrometric Methods.” Journal of pharmaceutical sciences vol. 105, 5 (2016): 1684-1692. doi:10.1016/j.xphs.2016.02.033.
[7] Danaei, M. M. Dehghankhold, S. Ataei, F. Hasanzadeh Davarani, R. Javanmard, A. Dokhani, S. Khorasani and M. R. Mozafari. Impact of Particle Size and Polydispersity Index on the Clinical Applications of Lipidic Nanocarrier Systems. Pharmaceutics. 10 (57) (2018) 1-17.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences that reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A particle comprising a plurality of platelet binding peptide (PBP) conjugates, von Willebrand factor-binding peptide (VBP) conjugates, or collagen-binding peptide (CBP) conjugates, or a combination thereof, wherein the PBP conjugate is a fibrinogen mimetic peptide (FMP) conjugate or a P-selectin binding peptide conjugate, and wherein the plurality of PBP conjugates, VBP conjugates, and/or CBP conjugates are conjugated to an outer surface of the particle, wherein the PBP conjugates, VBP conjugates, and CBP conjugates, taken together, are present at <5 molar percent of the particle.

2. The particle of claim 1, wherein the PBPs, VBPs, and/or CBPs or combination thereof, are spatially or topographically arranged on the particle surface such that the PBPs, VBPs, and/or CBPs or combinations thereof, do not spatially mask each other.

3. The particle of any one of claims 1-2, wherein the PBPs, VBPs, and/or CBPs or combination thereof, are conjugated to the particle surface with PEG linkers.

4. The particle of any one of claims 1-3, wherein the particle comprises DSPE-PEG2k-PBP, DSPE-PEG2k-VBP, or DSPE-PEG2k-CBP.

5. The particle of any one of claims 1-4, wherein the PBP conjugate comprises DSPE-PEG2k-FMP, wherein the FMP is of Formula (I):

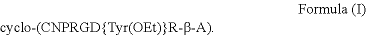

6. The particle of any one of claims 1-5, wherein the PBP conjugate comprises the amino acid sequence of SEQ ID NO:2.

7. The particle of any one of claims 1-5, wherein the PBP conjugate comprises the amino acid sequence of SEQ ID NO:3.

8. The particle of any one of claims 1-6, wherein the PBP conjugate comprises the amino acid sequence of SEQ ID NO:4.

9. The particle of any one of claims 1-7, wherein the PBP conjugate comprises the amino acid sequence of SEQ ID NO:5.

10. The particle of any one of claims 5-9, wherein the PBP is flanked by additional amino acid sequences.

11. The particle of any one of claims 1-10, wherein the VBPs comprise SEQ ID NO: 6.

12. The particle of claim 11, wherein the VBP is flanked by additional amino acid sequences.

13. The particle of any one of claims 1-12, wherein the CBPs comprise SEQ ID NO: 7.

14. The particle of claim 13, wherein the CBP is flanked by additional amino acid sequences.

15. The particle of any one of claims 1-14, wherein the particle comprises the plurality of PBP conjugates, a plurality of VBP conjugates, and a plurality of CBP conjugates.

16. The particle of any one of claims 1-15, wherein the molar percentage of PBP conjugate is between 0.05% and 5.00% molar percent of the particle.

17. The particle of any one of claims 1-16, wherein the molar percentage of FMP conjugate is between 0.05% and 5.00% molar percent of the particle.

18. The particle of any one of claims 1-17, wherein the molar percentage of CBP conjugate is between 0.10% and 1.50% molar percent of particle.

19. The particle of any one of claims 1-18, wherein the molar percentage of VBP conjugate is between 0.10% and 1.50% molar percent of the particle.

20. The particle of any one of claims 1-19, wherein the molar percentage of PBP conjugate is about 1.00% of the particle, the molar percentage of CBP conjugate is about 0.50% of the particle, and the molar percentage of VBP conjugate is about 0.50% of the particle.

21. The particle of any one of claims 1-20, wherein the particle comprises both VBP conjugates and CBP conjugates; and the ratio of VPBs to CBPs provided on the particle surface is about 70:30 to about 30:70.

22. The particle of any one of claims 1-21, wherein the relative molar ratios of PBP:CBP:VBP are 2:1:1.

23. The particle of any one of claims 1-21, wherein the relative molar ratios of PBP:CBP:VBP are 1:5:5.

24. The particle of any one of claims 1-21, wherein the relative molar ratios of PBP:CBP:VBP are 10:5:1.

25. The particle of any one of claims 1-21, wherein the relative molar ratios of PBP:CBP:VBP are 10:1:5.

26. The particle of any one of claims 1-21, wherein the relative molar ratios of PBP:CBP:VBP are 1:1:1.

27. The particle of any one of claims 1-21, wherein the relative molar ratios of PBP:CBP:VBP are 1:2:1.

28. The particle of any one of claims 1-21, wherein the relative molar ratios of PBP:CBP:VBP are 1:1:2.

29. The particle of any one of claims 1-21, wherein the relative molar ratios of PBP:CBP:VBP are 10:1:1.

30. The particle of any one of claims 1-10, wherein no CBP and/or VBP is conjugated to the surface of the particle.

31. The particle of any one of claims 1-10, wherein the relative molar ratios of PBP:CBP are 2:1.

32. The particle of any one of claims 1-10, wherein the relative molar ratios of PBP:VBP are 2:1.

33. The particle of any one of claims 1-32 further comprising a lipid.

34. The particle of claim 33, wherein the lipid comprises a phospholipid.

35. The particle of claim 33, wherein the lipid comprises a phospholipid conjugated to polyethylene glycol (PEG).

36. The particle of claim 35, wherein the PEG conjugated to the phospholipid has an average molecular weight from about 500 Da to about 5500 Da.

37. The particle of claim 33, wherein the lipid comprises a cholesterol, distearoylphosphatidylcholine (DSPC), or 1,2-distearoyl-sn-glycero-3-phosphoethanolamine (DSPE).

38. The particle of any one of claims 1-37 in the form of a liposome.

39. The particle of any one of claims 1-38, wherein the particle has a diameter of about 1-1000 nm.

40. The particle of any one of claims 1-38, wherein the particle has a diameter of about 30-300 nm.

41. The particle of any one of claims 1-40, wherein the particle is substantially spherical.

42. The particle of any one of claims 1-41, wherein the particle has a polydispersity index less than or equal to 0.3.

43. The particle of any one of claims 1-42, wherein the particle has a net positive or net negative zeta potential.

44. The particle of any one of claims 1-42, wherein the particle has a surface charge <−5 mV.

45. The particle of any one of claims 1-44, wherein the particle is stable for at least 12 weeks under storage in aqueous media.

46. The particle of any one of claims 1-45, wherein the particle promotes platelet aggregation.

47. The particle of any one of claims 1-46, the particle binds to activated platelets.

48. The particle of any one of claims 1-47, wherein the particle binds to ADP- or TRAP-activated platelets.

49. A fibrinogen mimetic peptide (FMP) of Formula (I): 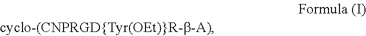
or a salt thereof.

50. The fibrinogen mimetic peptide of claim 49, wherein the fibrinogen mimetic peptide is an acetate salt of Formula (I), such as the trifluoroacetate salt of Formula (I).

51. The fibrinogen mimetic peptide of claim 49 or 50, wherein the fibrinogen mimetic peptide specifically binds to activated GPIIb-IIIa on platelets.

52. The fibrinogen mimetic peptide of any one of claims 49-51, wherein the fibrinogen mimetic peptide inhibits platelet aggregation.

53. The fibrinogen mimetic peptide of any one of claims 49-52, wherein the fibrinogen mimetic peptide has an IC₅₀ of about 0.13 μM.

54. A peptide conjugate comprising a peptide conjugated to a polymer, wherein the peptide is a fibrinogen mimetic peptide of any one of claims 49-53.

55. The peptide conjugate of claim 54, wherein the polymer is a lipid.

56. The peptide conjugate of claim 55, wherein the lipid comprises DSPE-PEG(2k).

57. The peptide conjugate of any one of claims 54-56, wherein the FMP is conjugated to the polymer through thio-ene coupling to the thiol group on the N-terminal cysteine.

58. A particle comprising a plurality of platelet binding peptide (PBP) conjugates, wherein the PBP conjugates comprise the peptide conjugates of any one of claims 55-57.

59. The particle of claim 58, further comprising a plurality of von Willebrand factor-binding peptide (VBP) conjugates or collagen-binding peptide (CBP) conjugates, or a combination thereof.

60. The particle of claim 58 or 59, wherein the PBPs, VBPs, and/or CBPs or combination thereof, are spatially or topographically arranged on the particle surface such that the PBPs, VBPs, and/or CBPs or combinations thereof, do not spatially mask each other.

61. The particle of any one of claims 58-60, wherein the PBPs, VBPs, and/or CBPs or combination thereof, are conjugated to the particle surface with PEG linkers.

62. The particle of any one of claims 58-61, wherein the particle comprises DSPE-PEG2k-PBP, DSPE-PEG2k-VBP, or DSPE-PEG2k-CBP.

63. The particle of any one of claims 59-62, wherein the PBP conjugate further comprises the amino acid sequence of SEQ ID NO:3.

64. The particle of any one of claims 59-62, wherein the PBP conjugate further comprises the amino acid sequence of SEQ ID NO:4.

65. The particle of any one of claims 59-62, wherein the PBP conjugate further comprises the amino acid sequence of SEQ ID NO:5.

66. The particle of any one of claims 58-65, wherein the PBP is flanked by additional amino acid sequences.

67. The particle of any one of claims 59-66, wherein the VBPs comprise SEQ ID NO: 6.

68. The particle of claim 67, wherein the VBP is flanked by additional amino acid sequences.

69. The particle of any one of claims 59-68, wherein the CBPs comprise SEQ ID NO: 7.

70. The particle of claim 69, wherein the CBP is flanked by additional amino acid sequences.

71. The particle of any one of claims 59-70, wherein the particle comprises the plurality of PBP conjugates, a plurality of VBP conjugates, and a plurality of CBP conjugates.

72. The particle of any one of claims 58-71, wherein the molar percentage of PBP conjugate is between 0.05% and 5.00% molar percent of the particle.

73. The particle of any one of claims 58-71, wherein the molar percentage of FMP conjugate is between 0.05% and 5.00% molar percent of the particle.

74. The particle of any one of claims 59-73, wherein the molar percentage of CBP conjugate is between 0.10% and 1.50% molar percent of the particle.

75. The particle of any one of claims 59-74, wherein the molar percentage of VBP conjugate is between 0.10% and 1.50% molar percent of the particle.

76. The particle of any one of claims 59-75, wherein the molar percentage of PBP conjugate is about 1.00% of the particle, the molar percentage of CBP conjugate is about 0.50% of the particle, and the molar percentage of VBP conjugate is about 0.50% of the particle.

77. The particle of any one of claims 59-76, wherein the particle comprises both VBP conjugates and CBP conjugates; and the ratio of VPBs to CBPs provided on the particle surface is about 70:30 to about 30:70.

78. The particle of any one of claims 59-77, wherein the relative molar ratios of PBP:CBP:VBP are 2:1:1.

79. The particle of any one of claims 59-75, wherein the relative molar ratios of PBP:CBP:VBP are 1:5:5.

80. The particle of any one of claims 59-75, wherein the relative molar ratios of PBP:CBP:VBP are 10:5:1.

81. The particle of any one of claims 59-75, wherein the relative molar ratios of PBP:CBP:VBP are 10:1:5.

82. The particle of any one of claims 59-75, wherein the relative molar ratios of PBP:CBP:VBP are 1:1:1.

83. The particle of any one of claims 59-75, wherein the relative molar ratios of PBP:CBP:VBP are 1:2:1.

84. The particle of any one of claims 59-75, wherein the relative molar ratios of PBP:CBP:VBP are 1:1:2.

85. The particle of any one of claims 59-75, wherein the relative molar ratios of PBP:CBP:VBP are 10:1:1.

86. The particle of any one of claims 59-66, wherein no CBP or no VBP is conjugated to the surface of the particle.

87. The particle of any one of claims 59-66, wherein the relative molar ratios of PBP:CBP are 2:1.

88. The particle of any one of claims 59-66, wherein the relative molar ratios of PBP:VBP are 2:1.

89. The particle of any one of claims 59-88 further comprising a lipid.

90. The particle of claim 89, wherein the lipid comprises a phospholipid.

91. The particle of claim 89, wherein the lipid comprises a phospholipid conjugated to polyethylene glycol (PEG).

92. The particle of claim 91, wherein the PEG conjugated to the phospholipid has an average molecular weight from about 500 Da to about 5500 Da.

93. The particle of claim 89, wherein the lipid comprises a cholesterol, distearoylphosphatidylcholine (DSPC), or 1,2-distearoyl-sn-glycero-3-phosphoethanolamine (DSPE).

94. The particle of any one of claims 58-93 in the form of a liposome.

95. The particle of any one of claims 58-94, wherein the particle has a diameter of about 1-1000 nm.

96. The particle of any one of claims 58-94, wherein the particle has a diameter of about 30-300 nm.

97. The particle of any one of claims 58-96, wherein the particle is substantially spherical.

98. The particle of any one of claims 58-97, wherein the particle has a polydispersity index less than or equal to 0.3.

99. The particle of any one of claims 58-98, wherein the particle has a net positive or net negative zeta potential.

100. The particle of any one of claims 58-98, wherein the particle has a surface charge <−5 mV.

101. The particle of any one of claims 58-100, wherein the particle is stable for at least 12 weeks under storage in aqueous media.

102. The particle of any one of claims 58-101, wherein the particle promotes platelet aggregation.

103. The particle of any one of claims 58-102, the particle binds to activated platelets.

104. The particle of any one of claims 58-103, wherein the particle binds to ADP- or TRAP-activated platelets.

105. A pharmaceutical composition comprising the fibrinogen mimetic peptide of any one of claims 49-53 and a carrier.

106. A pharmaceutical composition comprising the peptide conjugate of any one of claims 54-57 and a carrier.

107. A pharmaceutical composition comprising the particle of any one of claims 1-48 and 58-104 and a carrier.

108. The pharmaceutical composition of any one of claims 105-107, further comprising a therapeutic agent for the treatment of a disease or a condition.

109. The pharmaceutical composition of claim 108, wherein the particle encapsulates the therapeutic agent.

110. The pharmaceutical composition of claim 108 or 109, wherein the therapeutic agent is an antimicrobial, antiviral, or antifungal agent.

111. The pharmaceutical composition of claim 108 or 109, wherein the therapeutic agent is an anticancer agent.

112. The pharmaceutical composition of claim 108 or 109, wherein the therapeutic agent is a hemostatic agent.

113. The pharmaceutical composition of any one of claims 105-112, wherein the carrier is a pharmaceutically acceptable carrier.

114. A method of preventing or inhibiting platelet aggregation in a subject in need thereof, comprising administering to the subject a composition comprising the fibrinogen mimetic peptide (FMP) of any one of claims 49-53.

115. A method of preventing or inhibiting platelet aggregation in a subject in need thereof, the method comprising administering to the subject the fibrinogen mimetic peptide of any one of claims 49-53 or the pharmaceutical composition of claim 105.

116. A method of promoting aggregation of activated platelets on a site with exposed vWF and collagen, comprising administering to the site the particle of any one of claims 1-48 and 58-104, or the pharmaceutical composition of any one of claims 107-113.

117. A method of diminishing bleeding in a subject, comprising administering to a site of bleeding the particle of any one of claims 1-48 and 58-104, or the pharmaceutical composition of any one of claims 107-113.

118. A method of treating a vascular injury in a subject, comprising administering to a site of the vascular injury in the subject the particle of any one of claims 1-48 and 58-104, or the pharmaceutical composition of any one of claims 107-113.

119. The method of claim 118, wherein the vascular injury is a noncompressible hemorrhage due to trauma, bleeding due to surgery, or thrombocytopenia.

120. A method of promoting hemostasis in a subject in need thereof, comprising administering to the subject the particle of any one of claims 1-48 and 58-104, or the pharmaceutical composition of any one of claims 107-113.

121. A method of treating cancer in a subject in need thereof, comprising administering to the subject the pharmaceutical composition of claim 111.

122. The method of claim 121, wherein the subject has or is at risk of thrombocytopenia.
